(19) United States
(12) Patent Application Publication
(10) Pub. No.: US 2021/0135117 A1
(43) Pub. Date: May 6, 2021

(54) COMPOUND, LIGHT EMITTING MATERIAL AND ORGANIC LIGHT EMITTING ELEMENT

(71) Applicant: KYOTO UNIVERSITY, Sakyo-ku, Kyoto-shi, Kyoto (JP)

(72) Inventors: Hironori KAJI, Sakyo-ku, Kyoto-shi, Kyoto (JP); Yoshimasa WADA, Sakyo-ku, Kyoto-shi, Kyoto (JP); Shosei KUBO, Sakyo-ku, Kyoto-shi, Kyoto (JP)

(21) Appl. No.: 16/611,972

(22) PCT Filed: May 8, 2018
(86) PCT No.: PCT/JP2018/017704

§ 371 Date: Nov. 8, 2019

(30) Foreign Application Priority Data
May 8, 2017 (JP) 2017-092244

(51) Int. Cl. H01L 51/00; C09K 11/06; C09K 11/02; C07D 401/10

(52) U.S. Cl. CPC H01L 51/0067; C09K 11/06; C09K 11/02; C07D 401/10; H01L 51/5012; H01L 51/0052; C09K 2211/1018; C09K 2211/1007; C09K 2211/1011; H01L 51/0072

(57) ABSTRACT

A compound represented by the following formula (1a) or formula (1b) is a light emitting material capable of forming a film according to a coating method and excellent in heat resistance. R¹ and R² each are an aliphatic group, and at least one is a polycyclic aliphatic group. A¹ to A² each are N or C—R³, and R³ is a hydrogen atom or a substituent, at least one of A¹ to A⁵ is C—R³ and R is a donor group. A⁶ to A⁸ each are N or C—R⁴, and R⁴ represents a hydrogen atom or an alkyl group. At least one of A⁶ to A⁸ is N. D is a donor-like linking group.

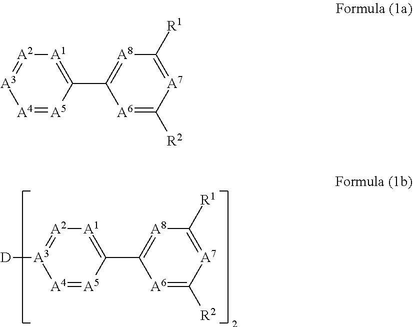

### TECHNICAL FIELD

The present invention relates to a compound useful as a light emitting material and to an organic light emitting device using the compound.

### BACKGROUND ART

An organic light emitting material not containing an inorganic atom has a high degree of freedom in molecular design, and therefore the properties thereof can be controlled varyingly. In addition, the material of the type is composed of a carbon atom, a hydrogen atom, a nitrogen atom and the like existing inexhaustibly in nature, and therefore has another advantage that the raw material cost for it is low. Consequently, special attention is paid to such an organic light emitting material not containing an inorganic atom.

In the case where a light emitting layer of a light emitting device is formed using such an organic light emitting material, a vacuum evaporation method is generally employed. However, a vacuum evaporation method requires a large-scaled vacuum evaporation apparatus, and in vacuum evaporation deposition, evaporant particles may adhere to any other area than a substrate, therefore causing a waste of raw materials. Consequently, when a light emitting device is produced according to a vacuum evaporation method, there occurs a problem that the production cost is high. In addition, particularly, in the case where a large-area light emitting layer to be applied to large-sized displays is formed, it is difficult to all over uniformly control the film thickness, and therefore there is another problem that defective products not having a uniform thickness are produced to lower the production yield.

Given the situation, investigations are being made for introducing a coating method into a production process for a light emitting device, as a film formation method capable of efficiently forming a film using a simple apparatus and capable of all over readily controlling the film thickness even in forming a large-area light emitting layer (for example, see PTLs 1 and 2).

### CITATION LIST

### Patent Literature

### PTL 1: JP 2015-072915A

### PTL 2: JP 2009-158535A

### SUMMARY OF INVENTION

### Technical Problem

Here, for forming a film of an organic light emitting material according to a coating method, it is necessary to prepare a solution of the organic light emitting material then apply this onto a substrate and dry it. However, organic light emitting materials heretofore proposed in the art have a low solubility in organic solvent and may form fine crystals in the process of drying the coating film, and consequently, many of them fail to be formed into films according to a coating method. Accordingly, when a light emitting layer is formed using an organic light emitting material, it is often inevitable to employ a dry process such as a vacuum evaporation method, and at present, the above-mentioned problems of cost increase and yield reduction are inevitable.

In addition, many of the organic light emitting materials that have been heretofore proposed in the art are poor in heat resistance, and are therefore problematic in point of safety in use for organic light emitting devices that may be kept under high-temperature conditions such as inside cars in summer season (for example, organic light emitting devices to be applied to car navigation systems, head-up displays or outdoor advertising displays, as well as to organic white illumination devices).

As described above, organic light emitting materials heretofore proposed in the art are not always satisfactory in point of practical use, for example in point of coating aptitude and heat resistance.

Accordingly, the present inventors have further promoted assiduous studies for the purpose of providing a novel light emitting material capable of readily forming a film according to a coating method by dissolving it in an organic solvent and excellent in heat resistance. Further, the present inventors have also promoted assiduous studies for the purpose of providing a highly practicable organic light emitting device that may be efficiently produced according to a coating method.

### Solution to Problem

As a result of promoting assiduous studies, the present inventors have found that a compound having a specific structure has a light emitting property and can form a film according to a coating method and is additionally excellent in heat resistance, and have herein provided the present invention as described below.

[1] A compound represented by the following formula (1a) or formula (1b):

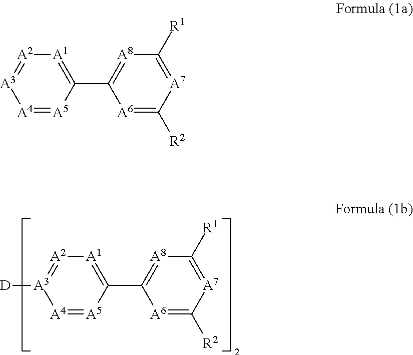

wherein R¹ and R² each independently represent an aliphatic group, and at least one represents a polycyclic aliphatic group; A¹ to A⁵ each independently represent N or C—R³, and R³ represents a hydrogen atom or a substituent, provided that at least one of A¹ to A⁵ is C—R³ and R³ is a donor group; A⁶ to A⁸ each independently represent N or C—R⁴, and R⁴ represents a hydrogen atom or an alkyl group, provided that at least one of A⁶ to A⁸ is N. In the formula (1b), D represents a donor-like linking group, and two R¹'s, two R²'s, and two A¹'s to two A¹'s existing in the formula each may be the same as or different from each other.
[2] The compound according to [1], wherein the polycyclic aliphatic group is a cage-structure aliphatic group.
[3] The compound according to [1] or [2], wherein R¹ and R² are both a polycyclic aliphatic group.
[4] The compound according any one of [1] to [3], wherein at least one R³ has a structure represented by the following formula (2):

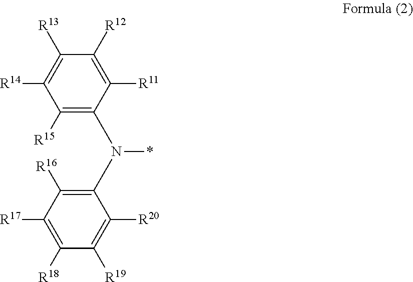

wherein R¹¹ to R²⁰ each independently represent a hydrogen atom or a substituent; R¹¹ and R¹², R¹² and R¹³, R¹³ and R¹⁴, R¹⁴ and R¹⁵, R¹⁵ and R¹⁶, R¹⁶ and R¹⁷, R¹⁷ and R¹⁸, R¹⁸ and R¹⁹, and R¹⁹ and R²⁰ each may bond to each other to form a cyclic structure; and * represents a bonding position.
[5] The compound according to [4], wherein the structure represented by the formula 2 has a structure represented b an of the following formulae (3) to (7):

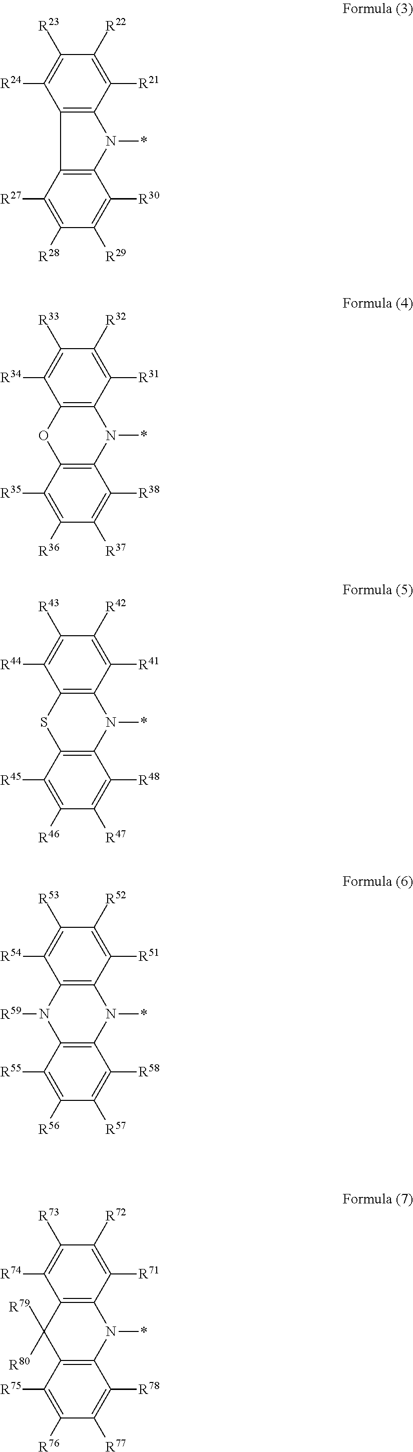

wherein R²¹ to R²⁴, R²⁷ to R³⁸, R⁴¹ to R⁴⁸, R⁵¹ to R⁵⁹, and R⁷¹ to R⁸⁰ each independently represent a hydrogen atom or a substituent; R²¹ and R²², R²² and R²³, R²³ and R²⁴, R²⁷ and R²⁸, R²⁸ and R²⁹, R²⁹ and R³⁰, R³¹ and R³², R³² and R³³, R³³ and R³⁴, R³⁵ and R³⁶, R³⁶ and R³⁷, R³⁷ and R³⁸, R⁴¹ and R⁴², R⁴² and R⁴³, R⁴³ and R⁴⁴, R⁴⁵ and R⁴⁶, R⁴⁶ and R⁴⁷, R⁴⁷ and R⁴⁸, R⁵¹ and R⁵², R⁵² and R⁵³, R⁵³ and R⁵⁴, R⁵⁵ and R⁵⁶, R⁵⁶ and R⁵⁷, R⁵⁷ and R⁵⁸, R⁵⁴ and R⁵⁹, R⁵⁵ and R⁵⁹, R⁷¹ and R⁷², R⁷² and R⁷³, R⁷³ and R⁷⁴, R⁷⁵ and R⁷⁶, R⁷⁶ and R⁷⁷, R⁷⁷ and R⁷⁸, and R⁷⁹ and R⁸⁰ each may bond to each other to form a cyclic structure; and * represents a bonding position.
[6] The compound according to any of [1] to [5], wherein, when A² is C—R³ (where R³ is a donor group), both A¹ and A³ are not N, when A³ is C—R³ (where R³ is a donor group), both A² and A⁴ are not N, and when A⁴ is C—R³ (where R³ is a donor group), both A³ and A⁵ are not N.
[7] The compound according to any of [1] to [6], wherein A¹ to A⁵ are all C—R³.
[8] The compound according to any of [1] to [7], wherein A⁶ to A⁸ are all N.
[9] The compound according to any of [1] to [8], which is composed of only a carbon atom, a nitrogen atom and a hydrogen atom.
[10] The compound according to any of [1] to [9], of which the difference ΔE_{ST} between the lowest excited singlet energy level and the lowest excited triplet energy level is 0.3 eV or less.
[11] A light emitting material containing a compound of any one of [1] to [10].
[12] A delayed fluorescent material containing a compound of any one of [1] to [10].
[13] An organic light emitting device containing a compound of any one of [1] to [10].
[14] The organic light emitting device according to [13], which is an organic electroluminescent device.
[15] The organic light emitting device according to [13] or [14], containing the compound in the light emitting layer therein.
[16] The organic light emitting device according to [15], wherein the light emitting layer contains a host material.
[17] The organic light emitting device according to any one of [13] to [14], which emits delayed fluorescence.
[18] The organic light emitting device according to any one of [13] to [17], which emits light such that the chromaticity coordinate x in the CIE-XYZ color coordinate system is 0.16 or less and y is 0.22 or less.

### Advantageous Effects of Invention

The compound of the present invention is a light emitting compound and can readily form a film according to a coating method by dissolving it in an organic solvent, and is excellent in heat resistance. Consequently, the compound of the present invention is highly practicable and is useful as a light emitting material. An organic light emitting device using the compound of the present invention as a light emitting material therein can be efficiently produced according to a coating method and has high heat resistance.

### BRIEF DESCRIPTION OF DRAWINGS

FIG. 1 This is a schematic cross-sectional view showing a layer configuration example of an organic electroluminescent device.

FIG. 2 This shows emission spectra of thin films of a compound 2 and any of compounds H1, H3 to H5.

FIG. 3 This shows graphs of a relationship between a doping concentration of a compound 2 and a photoluminescent quantum yield Φ_{PL}.

FIG. 4 This shows emission spectra of a thin film of a compound 1 and a compound H3, and a thin film of a compound H3 alone.

FIG. 5 This shows transient decay curves in light emission of thin films of a compound 1 and a compound H3.

FIG. 6 This is an emission spectrum of an organic electroluminescent device using a thin film of a compound 1 and a compound H3 as a light emitting layer.

FIG. 7 This is a graph of current density-voltage-luminance characteristic of an organic electroluminescent device using a co-deposited film of a compound 1 and a compound H3 as a light emitting layer.

FIG. 8 This is a graph of external quantum efficiency-current density characteristic of an organic electroluminescent device using a co-deposited film of a compound 1 and a compound H3 as a light emitting layer.

### DESCRIPTION OF EMBODIMENTS

The contents of the invention will be described in detail below. The constitutional elements may be described below with reference to representative embodiments and specific examples of the invention, but the invention is not limited to the embodiments and the examples. In the description herein, a numerical range expressed as “to” means a range that includes a numerical range described in the front and the rear of “to” as the upper limit and the lower limit. The hydrogen atom that is present in the molecules of the compound used in the invention is not particularly limited in isotope species, and for example, all the hydrogen atoms in the molecule may be ¹H, and all or a part of them may be ²H (deuterium (D)).

[Compound Represented by Formula (1a) or Formula (1b)]

The light emitting material of the present invention contains a compound represented by the following formula (1a) or formula (1b).

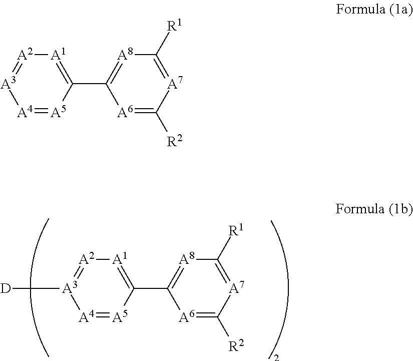

In the formulae (1a) and (1b), A¹ to A⁵ each independently represent N or C—R³, and R³ represents a hydrogen atom or a substituent. However, at least one of A¹ to A⁵ is C—R³ and R³ is a donor group. Any one or any two or more of A¹ to A⁵ may be C—R³ where R³ is a donor group, but preferably 1 to 3 of them, more preferably one or two of them, and even more preferably one of them is C—R³ where R³ is a donor group. Among A¹ to A⁵, preferably, at least one of A² to A⁴ is C—R³ where R³ is a donor group, and more preferably at least A³ is C—R³ where R³ is a donor group. When 2 or more of A¹ to A⁵ each are C—R³ where R³ is a donor group, multiple donor groups may be the same as or different from each other. In the formula (1b) two A¹'s to A⁵'s existing in the molecule each may be the same as or different from each other.

The “donor group” in the present invention means a group that donates an electron to an atomic group to which the donor group bonds. For example, the group may be selected from substituents having a negative Hammett's σₚ value.

Here, “Hammett's σₚ value” is one propounded by L. P. Hammett, and is one to quantify the influence of a substituent on the reaction rate or the equilibrium of a para-substituted benzene derivative. Specifically, the value is a constant (σₚ) peculiar to the substituent in the following equation that is established between a substituent and a reaction rate constant or an equilibrium constant in a para-substituted benzene derivative:

log(k/k₀)=ρσₚ

or

log(k/k₀)=ρσₚ

In the above equations, k represents a rate constant of a benzene derivative not having a substituent; k₀ represents a rate constant of a benzene derivative substituted with a substituent; K represents an equilibrium constant of a benzene derivative not having a substituent; K₀ represents an equilibrium constant of a benzene derivative substituted with a substituent; ρ represents a reaction constant to be determined by the kind and the condition of reaction. Regarding the description relating to the “Hammett's σₚ value” and the numerical value of each substituent, reference may be made to the description relating to σₚ value in Hansch, C. et. al., Chem. Rev., 91, 165-195 (1991).

As the donor group, preferably employed is an electron-donating substituent bonding at a hetero atom selected from the group consisting of a nitrogen atom, an oxygen atom, a sulfur atom, a silicon atom and a phosphorus atom, or an electron-donating aryl group. An electron-donating aryl group is generally a substituted aryl group and is preferably an aryl group substituted with an electron-donating substituent bonding at a hetero atom selected from the group consisting of a nitrogen atom, an oxygen atom, a sulfur atom, a silicon atom and a phosphorus atom.

Also preferably, the donor group contains a substituted or unsubstituted diarylamino structure, and is more preferably an aryl group substituted with a substituted or unsubstituted diarylamino group. Here, “diarylamino structure” means both a diarylamino group and a heteroaromatic ring structure in which both the aryl groups in a diarylamino group bond together via a single bond or a linking group to form a hetero ring. The aromatic ring to constitute each aryl group of a diarylamino group, and the aromatic ring to constitute each aryl group of an aryl group substituted with a diarylamino group (each aryl group of the diarylamino group and the aryl group substituted with the diarylamino group) may be a monocyclic ring or a condensed ring of 2 or more aromatic rings condensed together, or may also be a linked ring of 2 or more aromatic rings linking together. In the case where 2 or more aromatic rings link together, they may link linearly or as a branched structure. The carbon number of the aromatic ring to constitute each aryl group of the diarylamino structure and the aryl group substituted with a diarylamino group is preferably 6 to 22, more preferably 6 to 18, even more preferably 6 to 14, and further more preferably 6 to 10. Specific examples of the aryl group include a phenyl group, a naphthyl group, and a biphenyl group. Regarding the description and the preferred range of the substituent in the case where the aryl group of the diarylamino structure and the aryl group substituted with a diarylamino group have a substituent, reference may be made to the description and the preferred range of the substituent that R¹¹ to R²⁰ mentioned below may have. Regarding the description and the preferred range of the linking group that links aryl groups together in the case where the diarylamino structure is the above-mentioned heteroaromatic ring structure, reference may be made to the description and the preferred range of the linking group in the case where R¹⁵ and R¹⁶ in the following formula (2) bond together to form a linking group.

The donor group is preferably a group represented by the following formula (2).

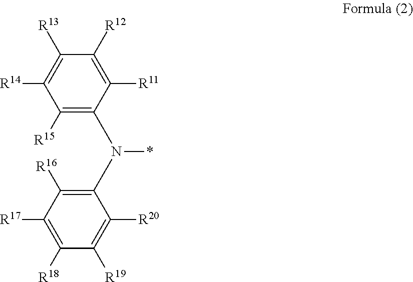

In the formula (2), R¹¹ to R²⁰ each independently represent a hydrogen atom or a substituent. The number of the substituents is not specifically limited, and all of R¹¹ to R²⁰ are unsubstituted (that is, hydrogen atoms). In the case where 2 or more of R¹¹ to R²⁰ are substituents, the multiple substituents may be the same as or different from each other. * represents a bonding position.

Examples of the substituent that R¹¹ to R²⁰ may have include a hydroxyl group, a halogen atom, an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 1 to 20 carbon atoms, an aryl group having 6 to 40 carbon atoms, a heteroaryl group having 3 to 40 carbon atoms, an alkenyl group having 2 to 10 carbon atoms, an alkynyl group having 2 to 10 carbon atoms, an alkylamide group having 2 to 20 carbon atoms, an arylamide group having 7 to 21 carbon atoms, and a trialkylsilyl group having 3 to 20 carbon atoms. Among these specific groups, those that can be further substituted with any substituent may be substituted. More preferred substituents include an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 1 to 20 carbon atoms, an aryl group having 6 to 40 carbon atoms, a heteroaryl group having 3 to 40 carbon atoms, and a group of the formula (1a) or the formula (1b) where one R³ is change to a single bond.

R¹¹ and R¹², R¹² and R¹³, R¹³ and R¹⁴, R¹⁴ and R¹⁵, R¹⁵ and R¹⁶, R¹⁶ and R¹⁷, R¹⁷ and R¹⁸, R¹⁸ and R¹⁹, and R¹⁹ and R²⁰ each may bond to each other to form a cyclic structure. The cyclic structure may be an aromatic ring or an aliphatic ring, and may contain a hetero atom, and further, the cyclic structure may be a condensed ring of 2 or more rings. Here, the hetero atom is preferably selected from the group consisting of a nitrogen atom, an oxygen atom and a sulfur atom. Examples of the cyclic structure to be formed include a benzene ring, a naphthalene ring, a pyridine ring, a pyridazine ring, a pyrimidine ring, a pyrazine ring, a pyrrole ring, an imidazole ring, a pyrazole ring, an imidazoline ring, an oxazole ring, an isoxazole ring, a thiazole ring, an isothiazole ring, a cyclohexadiene ring, a cyclohexene ring, a cyclopentaene ring, a cycloheptatriene ring, a cycloheptadiene ring, and a cycloheptaene ring.

Among the groups represented by the formula (2), preferred are those where R¹⁵ and R¹⁶ do not bond to each other, those where R¹⁵ and R¹⁶ bond to each other via a single bond, and those where R¹⁵ and R¹⁶ bond to each other to form a linking group in which the linking chain length is one atom. In the case where R¹⁵ and R¹⁶ bond to each other to form a linking group in which the linking chain length is one atom, the cyclic structure to be formed as a result of bonding of R¹⁵ and R¹⁶ to each other is a 6-membered ring. Specific examples of the linking group to be formed by bonding of R¹⁵ and R¹⁶ to each other includes a linking group represented by —O—, —S—, —N(R⁹¹)— or —C(R⁹²)(R⁹³)—. In these, R⁹¹ to R⁹³ each independently represent a hydrogen atom or a substituent. Examples of the substituent that R⁹¹ may have include an alkyl group having 1 to 20 carbon atoms, an aryl group having 6 to 40 carbon atoms, and a heteroaryl group having 3 to 40 carbon atoms. Examples of the substituent that R⁹² and R⁹³ may have each independently include a hydroxy group, a halogen atom, an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 1 to 20 carbon atoms, an aryl group having 6 to 40 carbon atoms, a heteroaryl group having 3 to 40 carbon atoms, an alkenyl group having 2 to 10 carbon atoms, an alkynyl group having 2 to 10 carbon atoms, an alkylamide group having 2 to 20 carbon atoms, an arylamide group having 7 to 21 carbon atoms and a trialkylsilyl group having 3 to 20 carbon atoms.

Preferred examples of the group represented by the formula (2) include groups represented by any of the following formulae (3) to (7).

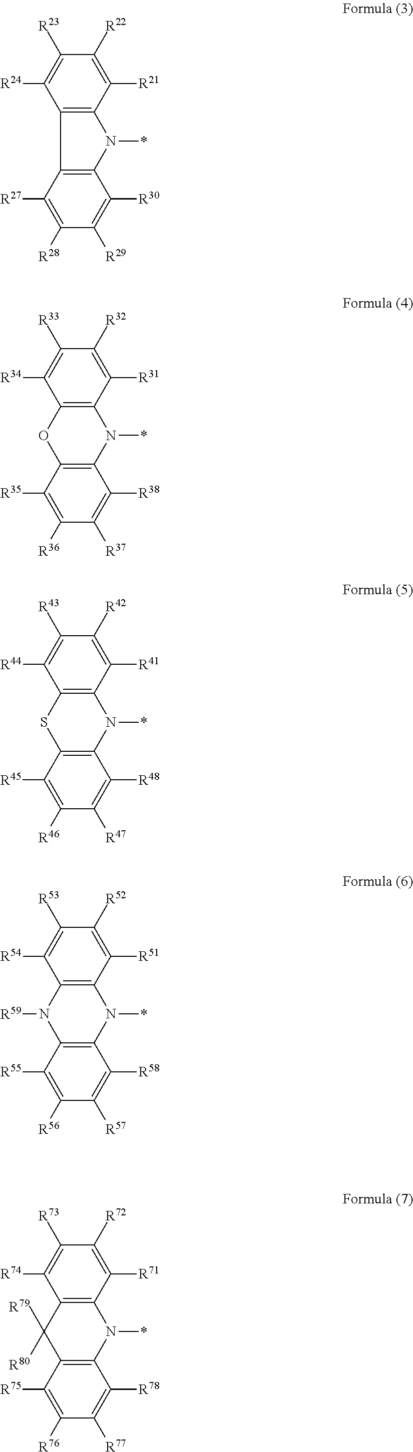

In the formulae (3) to (7), R²¹ to R²⁴, R²⁷ to R³⁸, R⁴¹ to R⁴⁸, R⁵¹ to R⁵⁹, and R⁷¹ to R⁸⁰ each independently represent a hydrogen atom or a substituent. Regarding the description and preferred ranges of the substituents as referred to herein, reference may be made to the description and the preferred ranges of the substituents that R¹¹ to R²⁰ may have as described above. Also preferably, R²¹ to R²⁴, R²⁷ to R³⁸, R⁴¹ to R⁴⁸, R⁵¹ to R⁵⁹, and R⁷¹ to R⁸⁰ each are independently a group represented by any of the formulae (3) to (7). * represents a bonding position. Preferably, R⁷⁹ and R⁸⁰ in the formula (7) each are a substituted or unsubstituted alkyl group, more preferably a substituted or unsubstituted alkyl group having 1 to 6 carbon atoms. Also preferably, R⁷⁹ and R⁸⁰ in the formula (7) each are a substituted or unsubstituted aryl group, more preferably a substituted or unsubstituted aryl group having 6 to 40 carbon atoms, even more preferably a substituted or unsubstituted aryl group having 6 to 10 carbon atoms, and especially more preferably a phenyl group. Further, when R⁷⁹ and R⁸⁰ in the formula (7) each are a substituted or unsubstituted aryl group, the aryl groups preferably may bond to each other to form a cyclic structure. The number of the substituents in the formulae (3) to (7) is not specifically limited. A case where all are unsubstituted (that is all are hydrogen atoms) is also preferred. In the case where the formulae (3) to (7) each have 2 or more substituents, the substituents may be the same as or different from each other. In the case where a substituent exists in the formulae (3) to (7) and when the substituent is the formula (3), the substituent is preferably any of R²² to R²⁴, and R²⁷ to R²⁹, more preferably at least one of R²³ and R²⁸; when the substituent is the formula (4), it is preferably any of R³² to R³⁷, when the substituent is the formula (5), it is preferably any of R⁴² to R⁴⁷; when the substituent is the formula (6), it is preferably any of R⁵², R⁵³, R⁵⁶, R⁵⁷, and R⁵⁹; and when the substituent is the formula (7), it is preferably any of R⁷² to R⁷⁷, R⁷⁹, and R⁸⁰.

In the formulae (3) to (7), R²¹ and R²², R²² and R²³, R²³ and R²⁴, R²⁷ and R²⁸, R²⁸ and R²⁹, R²⁹ and R³⁰, R³¹ and R³², R³² and R³³, R³³ and R³⁴, R³⁵ and R³⁶, R³⁶ and R³⁷, R³⁷ and R³⁸, R⁴¹ and R⁴², R⁴² and R⁴³, R⁴³ and R⁴⁴, R⁴⁵ and R⁴⁶, R⁴⁶ and R⁴⁷, R⁴⁷ and R⁴⁸, R⁵¹ and R⁵², R⁵² and R⁵³, R⁵³ and R⁵⁴, R⁵⁵ and R⁵⁶, R⁵⁶ and R⁵⁷, R⁵⁷ and R⁵⁸, R⁵⁴ and R⁵⁹, R⁵⁵ and R⁵⁹, R⁷¹ and R⁷², R⁷² and R⁷³, R⁷³ and R⁷⁴, R⁷⁵ and R⁷⁶, R⁷⁶ and R⁷⁷, R⁷⁷ and R⁷⁸, and R⁷⁹ and R⁸⁰ each may bond to each other to form a cyclic structure. Regarding the description and preferred examples of the cyclic structure, reference may be made to the description and the preferred examples of the cyclic structure to be formed by R¹¹ and R¹² and the like in the formula (2) bonding to each other.

Preferably, the group represented by the formula (6) includes groups represented by the following formula (6′).

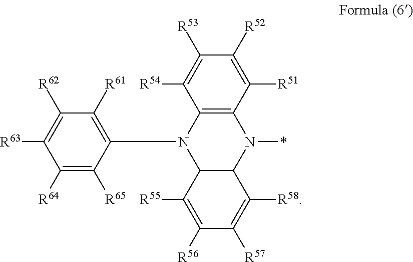

In the formula (6′), R⁵¹ to R⁵⁸, and R⁶¹ to R⁶⁵ each independently represent a hydrogen atom or a substituent. R⁵¹ and R⁵², R⁵² and R⁵³, R⁵³ and R⁵⁴, R⁵⁵ and R⁵⁶, R⁵⁶ and R⁵⁷, R⁵⁷ and R⁵⁸, R⁶¹ and R⁶², R⁶² and R⁶³, R⁶³ and R⁶⁴, R⁶⁴ and R⁶⁵, R⁵⁴ and R⁶¹, and R⁵⁵ and R⁶⁵ each may bond to each other to form a cyclic structure. * represents a bonding position.

Among A¹ to A⁵, the others than C—R³ (where R³ represents a donor group) each are N or C—R³ in which R³ is a hydrogen atom and any other substituent than a donor group. Here, R³ is preferably a hydrogen atom. When R³ is any other substituent than a donor group, the substituent is preferably one having a σₚ value of 1 or less, more preferably 0.5 or less, even more preferably 0.2 or less. The number of A¹ to A⁵ representing N may be 0 or may be 1 to 4, but is preferably 0 to 2, more preferably 0 or 1, even more preferably 0. Namely, it is preferable that all A¹ to A⁵ each are C—R³.

Preferably, A¹ to A⁵ satisfy the following requirement I. With that, the steric structure of the molecule can be twisted owing to the steric interaction between the donor group and R³ bonding to the carbon atom at both sides or one side of the atom to which the donor group bonds.

### (Requirement 1)

When A² is C—R³ (where R³ is a donor group), both A¹ and A³ are not N, and when A³ is C—R³ (where R³ is a donor group), both A² and A⁴ are not N, and when A⁴ is C—R³ (where R³ is a donor group), both A³ and A⁵ are not N.

In each of the formulae (1a) and (1b), A⁶ to A⁸ each independently represent N or C—R⁴, and R⁴ represents a hydrogen atom or an alkyl group, provided that at least one of A⁶ to A⁸ is N. One or two of A⁶ to A⁸ may be N, and all of A⁶ to A⁸ may be N, and preferably all of A⁶ to A⁸ are N. When one or two of A⁶ to A⁸ are N, the remaining group is C—R⁴. The alkyl group of R⁴ may be linear, branched or cyclic. Preferably, the alkyl group has 1 to 20 carbon atoms, more preferably 1 to 10 carbon atoms, even more preferably 1 to 6 carbon atoms. Examples of the alkyl group include a methyl group and an ethyl group. When two of A⁶ to A⁸ are C—R⁴, two R⁴'s may be the same as or different from each other. In the formula (1b), two A⁶'s to A⁸'s existing in the molecule each may be the same as or different from each other.

In each of the formulae (1a) and (1b), R¹ and R² each independently represent an aliphatic group, and at least one represents a polycyclic aliphatic group. In the formula (1b), two R¹'s and two R²'s existing in the molecule each may be the same as or different from each other.

In the present invention, the “aliphatic group” is a residue of a non-aromatic carbon compound, and may be one having a cyclic structure or may be one not having a cyclic structure. The aliphatic group may be an aliphatic hydrocarbon group derived from an aliphatic hydrocarbon by removing one hydrogen therefrom, or may be a hetero-aliphatic group formed by substituting the carbon atom or the hydrogen atom of an aliphatic hydrocarbon group with a hetero atom, but is preferably an aliphatic hydrocarbon group. The hetero atom to constitute a hetero-aliphatic group includes a nitrogen atom, an oxygen atom, a sulfur atom, a silicon atom and a phosphorus atom, but is preferably a nitrogen atom.

In the present invention, the “polycyclic aliphatic group” is an aliphatic group having an aliphatic polycyclic structure, and the “aliphatic polycyclic structure” is a structure formed by condensing two or more alicyclic ring with sharing two or more atoms therein. The aliphatic polycyclic structure to constitute a polycyclic aliphatic group may bond to the carbon atom of the 6-membered ring that has A⁶ to A⁸ in the formula (1a) or (1b) via single bond, or may bond thereto via a divalent aliphatic group such as an alkylene group, but preferably bonds to the carbon atom of the 6-membered ring that has A⁶ to A⁸ via a single bond. The number of the aliphatic polycyclic structures that the polycyclic structure has may be one, or two or more. In the case where the polycyclic aliphatic group has two or more aliphatic polycyclic structures, the aliphatic polycyclic structures may be the same as or different from each other. In this case, the neighboring aliphatic polycyclic structures may bond to each other via a single bond, or may bond via a divalent aliphatic group such as an alkylene group.

The aliphatic polycyclic structure to constitute a polycyclic aliphatic group includes a condensed ring structure, a crosslinked ring structure and a cage structure. The number of the rings of each monocyclic ring that constitutes the aliphatic polycyclic structure is preferably 3 to 8, more preferably 4 to 7, even more preferably 5 or 6. The number of the rings constituting the aliphatic polycyclic structure is preferably 2 to 10, more preferably 2 to 6, even more preferably 2 to 4. Among these, a preferred aliphatic polycyclic structure includes a cage structure, and a bicyclo ring and a tricyclo ring having a condensed ring structure or a crosslinked ring structure, and a more preferred aliphatic polycyclic structure is a cage structure. Especially preferably, the polycyclic aliphatic group is a cage-structure aliphatic group having a cage structure and configured such that any carbon atom constituting the cage structure bond to the carbon atom of the 6-membered ring having A⁶ to A⁸ via a single bond. Specific examples of the cage structure having alicyclic ring include adamantane, cubane, cuneane, carfene, fullerene (C60, C70, C84, etc.), and adamantane is preferred. The polycyclic aliphatic group having adamantane (adamantyl group) may be a 1-adamantyl group or a 2-adamantyl group, but is preferably a 1-adamantyl group. Specific examples of the aliphatic polycyclic structure mentioned herein may be substituted with a substituent. Regarding the description and preferred ranges of the substituent, reference may be made to the description and the preferred ranges of the substituents that R¹¹ to R²⁰ mentioned above may have, and among them, substituents not containing an aromatic ring are preferred.

Among R¹ and R², one of R¹ and R² may be a polycyclic aliphatic group or both R¹ and R² may be a polycyclic aliphatic group, but preferably both R¹ and R² are a polycyclic aliphatic group. When both R¹ and R² are a polycyclic aliphatic group, the polycyclic aliphatic groups may be the same as or different from each other.

When one of R¹ and R² is a polycyclic aliphatic group, the aliphatic group represented by the other may be linear, branched or monocyclic, and may have a structure formed of a linear or branched aliphatic chain and a monocyclic alicyclic ring bonding to each other. The carbon number of the aliphatic group represented by the other R¹ or R² is preferably 1 to 85, more preferably 1 to 40, even more preferably 1 to 20. Specific examples of the aliphatic group include an alkyl group having 1 to 20 carbon atoms, an alkenyl group having 2 to 10 carbon atoms, and an alkynyl group having 2 to 10 carbon atoms. Specific examples of the aliphatic group mentioned herein may be substituted with a substituent. Regarding the description and preferred ranges of the substituent, reference may be made to the description and the preferred ranges of the substituents that the above-mentioned R¹¹ to R²⁰ may have, and among them, preferred are substituents not containing an aromatic ring.

In the formula (1b), D represents a donor-like linking group.

The “donor-like linking group” in the present invention means a divalent group that links the two parenthesized structural units in the formula (1b) and imparts an electron to the atomic group to which the donor-like linking group bonds. Examples of the donor-like linking group include a divalent group having a structure of two donor groups bonding to each other. Regarding the description and preferred ranges of the donor group, reference may be made to the description and the preferred ranges of the “donor group” for R³ mentioned hereinabove. The divalent group having structure of two donor groups bonding to each other is, for example, a divalent group having a structure of two groups represented by the formula (2) bonding to each other in which any of R¹¹ to R¹⁹ of the group represented by the formula (2) is a linking position, or in a cyclic structure formed by R¹⁵ and R¹⁶ bonding to each other, a position substitutable with a hydrogen atom is a linking position. Here, the two groups represented by the formula (2) may be the same as or different from each other. The two groups represented by the formula (2) may bond to each other via a single bond or via a spiro bond, or via a linking group such as an arylene group, for example, a phenylene group, or an alkylene group. Preferred examples of the donor-like linking group include groups represented by the following formula (7a). Regarding the description of R⁷¹ to R⁷⁸ in the formula (7a), reference may be made to the description of R⁷¹ to R⁷⁸ in the formula (7). Two R⁷'s to R⁷'s existing in the donor-like linking group each may be the same as or different from each other.

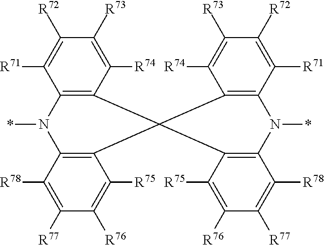

The compound represented by the formula (1a) or the formula (1b) may be a compound composed of only a carbon atom, a nitrogen atom and a hydrogen atom. For example, when the compound contains an atom that may readily impart polarity to the molecule thereof, such as a fluorine atom, a phosphorus atom or a sulfur atom, the solubility of the compound in an organic solvent may lower, but when the compound is composed of only a carbon atom, a nitrogen atom and a hydrogen atom, the compound exhibits good solubility in an organic solvent, and therefore according to a coating method using the compound, a film of the compound may be formed more easily.

The compound represented by the formula (1a) or the formula (1b) is preferably a compound such that the difference ΔEₛₜ, between the lowest excited singlet energy level SI and the lowest excited triplet energy level T1 of the compound is small. Specifically, ΔEₛₜ is preferably 0.3 eV or less, more preferably 0.2 eV or less, even more preferably 0.1 eV or less, and further more preferably 0.05 eV or less. ΔEₛₜ as referred to herein is calculated according to the method described on page 18077 in Q. Zhang el al., J. Am. Chem. Soc. 136, 18070 (2014) and from the equation (15) in the literature.

Examples of the compound represented by the formula (1a) or the formula (1b) include compounds each having a structure mentioned below. However, the compound represented by the formula (1a) or the formula (1b) usable in the present invention should not be limitatively interpreted by these specific examples.

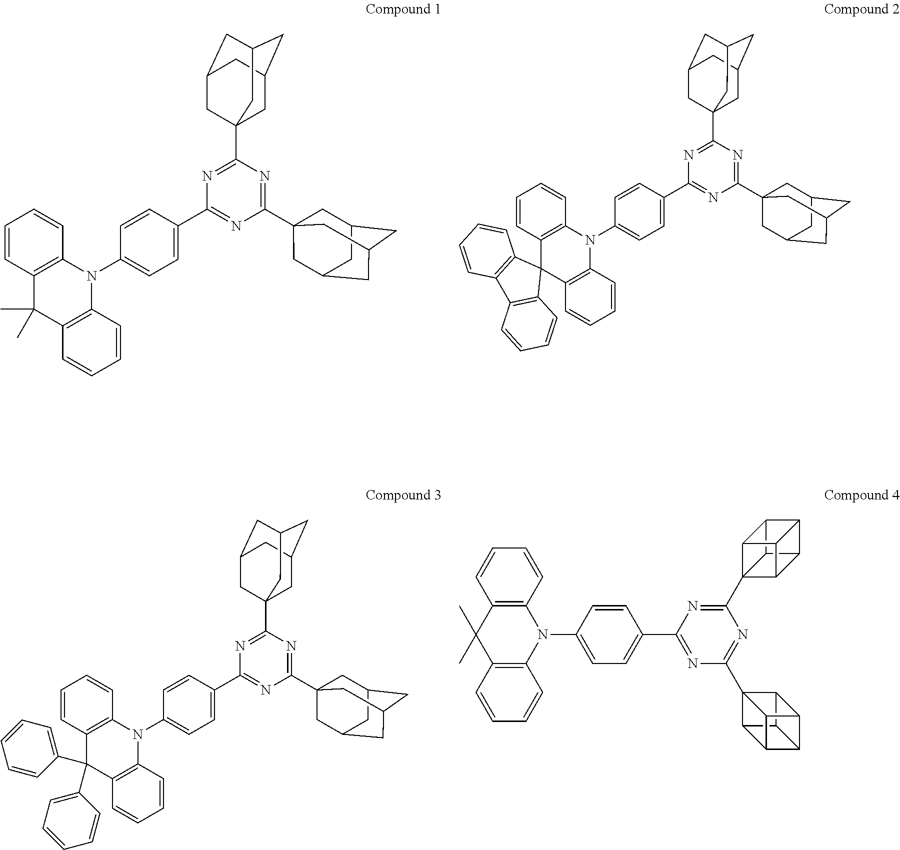 [SMILES: CC1(C)c2ccccc2N(c2ccc(-c3nc(C45CC6CC(CC(C6)C4)C5)nc(C45CC6CC(CC(C6)C4)C5)n3)cc2)c2ccccc21 ; c1ccc2c(c1)-c1ccccc1C21c2ccccc2N(c2ccc(-c3nc(C45CC6CC(CC(C6)C4)C5)nc(C45CC6CC(CC(C6)C4)C5)n3)cc2)c2ccccc21 ; c1ccc(C2(c3ccccc3)c3ccccc3N(c3ccc(-c4nc(C56CC7CC(CC(C7)C5)C6)nc(C56CC7CC(CC(C7)C5)C6)n4)cc3)c3ccccc32)cc1 ; CC1(C)c2ccccc2N(c2ccc(-c3nc(C45C6C7C8C6C4C8C75)nc(C45C6C7C8C6C4C8C75)n3)cc2)c2ccccc21]

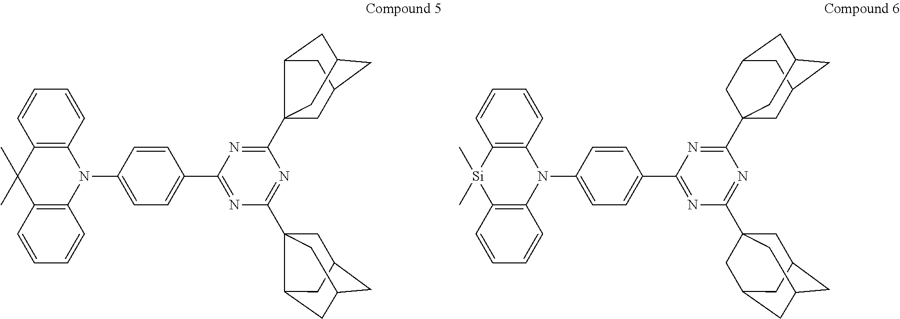 [SMILES: CC1(C)c2ccccc2N(c2ccc(-c3nc(C45CC6CC(CC4C6)C5)nc(C45CC6CC(CC4C6)C5)n3)cc2)c2ccccc21 ; C[Si]1(C)c2ccccc2N(c2ccc(-c3nc(C45CC6CC(CC(C6)C4)C5)nc(C45CC6CC(CC(C6)C4)C5)n3)cc2)c2ccccc21]

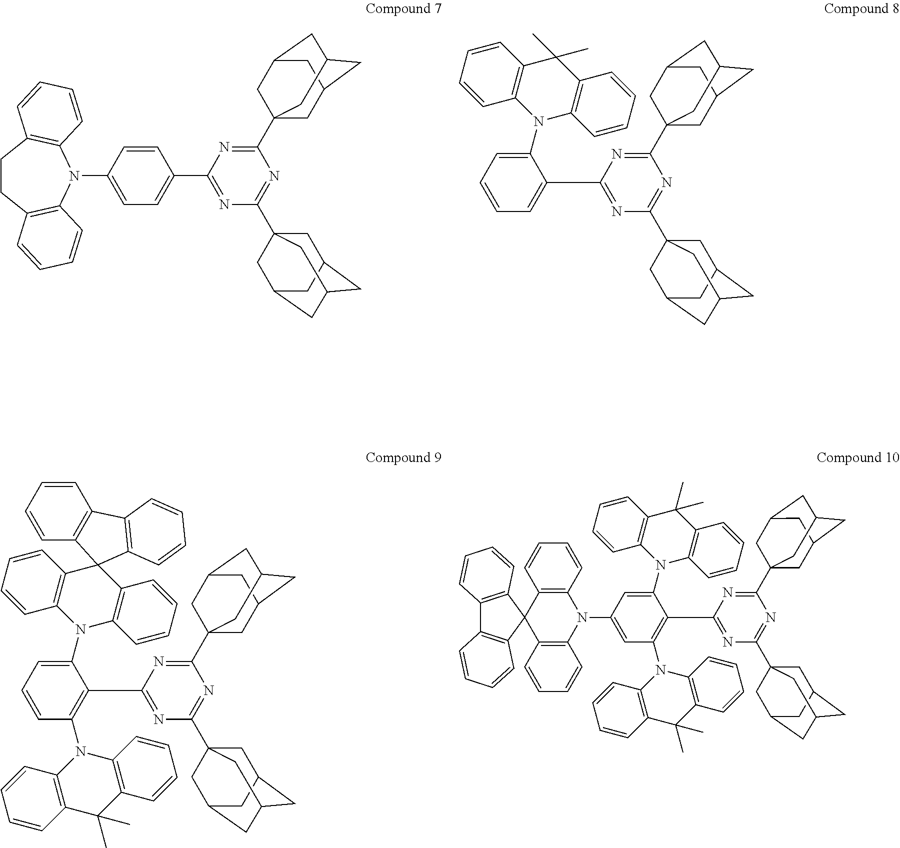 [SMILES: c1ccc2c(c1)CCc1ccccc1N2c1ccc(-c2nc(C34CC5CC(CC(C5)C3)C4)nc(C34CC5CC(CC(C5)C3)C4)n2)cc1 ; CC1(C)c2ccccc2N(c2ccccc2-c2nc(C34CC5CC(CC(C5)C3)C4)nc(C34CC5CC(CC(C5)C3)C4)n2)c2ccccc21 ; CC1(C)c2ccccc2N(c2cccc(N3c4ccccc4C4(c5ccccc5-c5ccccc54)c4ccccc43)c2-c2nc(C34CC5CC(CC(C5)C3)C4)nc(C34CC5CC(CC(C5)C3)C4)n2)c2ccccc21 ; CC1(C)c2ccccc2N(c2cc(N3c4ccccc4C4(c5ccccc5-c5ccccc54)c4ccccc43)cc(N3c4ccccc4C(C)(C)c4ccccc43)c2-c2nc(C34CC5CC(CC(C5)C3)C4)nc(C34CC5CC(CC(C5)C3)C4)n2)c2ccccc21]

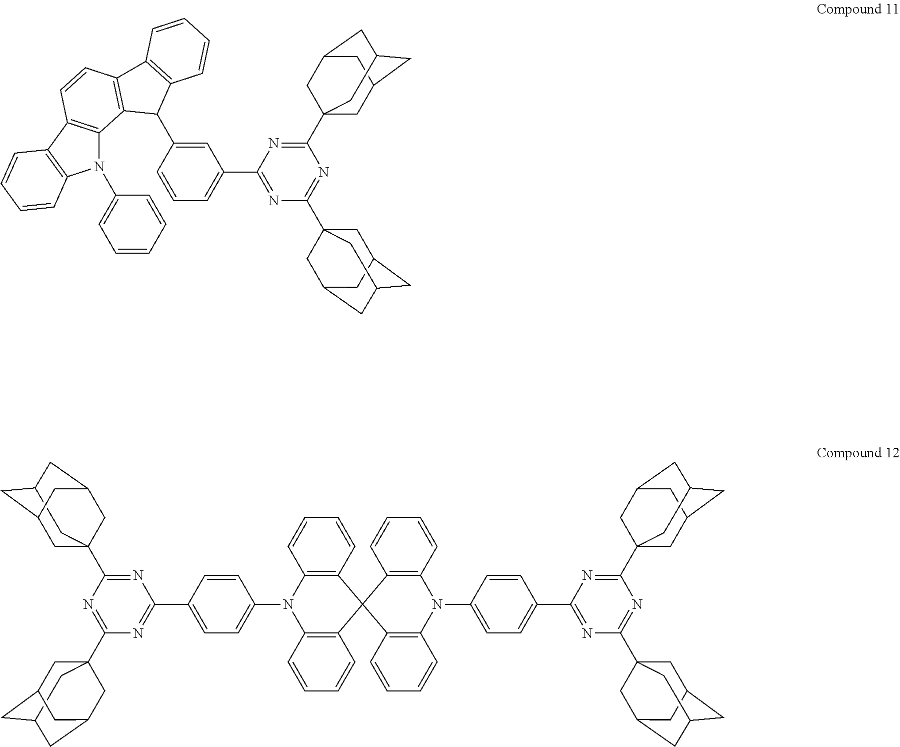 [SMILES: c1ccc(-n2c3ccccc3c3ccc4c(c32)C(c2cccc(-c3nc(C56CC7CC(CC(C7)C5)C6)nc(C56CC7CC(CC(C7)C5)C6)n3)c2)c2ccccc2-4)cc1 ; c1ccc2c(c1)N(c1ccc(-c3nc(C45CC6CC(CC(C6)C4)C5)nc(C45CC6CC(CC(C6)C4)C5)n3)cc1)c1ccccc1C21c2ccccc2N(c2ccc(-c3nc(C45CC6CC(CC(C6)C4)C5)nc(C45CC6CC(CC(C6)C4)C5)n3)cc2)c2ccccc21]

The molecular weight of the compound represented by the formula (1a) or the formula (1b) is, for example, in the case where an organic layer containing the compound represented by the formula (1a) or the formula (1b) is intended to be formed according to a vapor deposition method and used in devices, preferably 1500 or less, more preferably 1200 or less, even more preferably 1000 or less, and further more preferably 800 or less. The lower limit of the molecular weight is the smallest molecular weight that the formula (1a) can take, more preferably a molecular weight larger by 20 than the smallest molecular weight that the formula (1a) can take.

Irrespective of the molecular weight thereof, the compound represented by the formula (1a) or the formula (1b) may be formed into a film according to a coating method. The compound represented by the formula (1a) or the formula (1b) has an excellent coating aptitude irrespective of the molecular weight thereof, and can readily form a film according to a coating method.

Applying the present invention, it is considered to use a compound containing multiple structures represented by the formula (1a) or the formula (1b) in the molecule as a light emitting material.

For example, it is considered that a polymerizable group is previously introduced into a structure represented by the formula (1a) or the formula (1b) and the polymerizable group is polymerized to give a polymer, and the polymer is used as a light emitting material. Specifically, a monomer containing a polymerizable functional group in any of R¹ to R⁴ in the formula (1a) or in any of R¹ to R⁴, and D in the formula (1b) is prepared, and this is homo-polymerized or copolymerized with any other monomer to give a polymer having a recurring unit, and the polymer can be used as a light emitting material. Alternatively, compounds each having a structure represented by the formula (1a) or the formula (1b) are coupled to give a dimer or a trimer, and it can be used as a light emitting material.

Examples of the polymer having a recurring unit containing a structure represented by the formula (1a) or the formula (1b) include polymers containing a structure represented by the following formula (11) or (12).

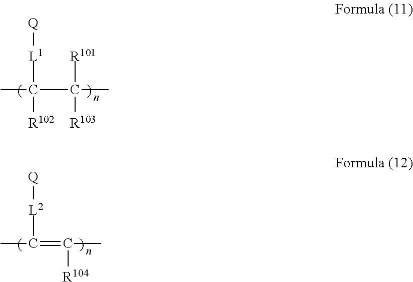

In the formula (11) or (12), Q represents a group containing a structure represented by the formula (1a) or the formula (1b), and L¹ and L² each represent a linking group. The carbon number of the linking group is preferably 0 to 20, more preferably 1 to 15, even more preferably 2 to 10. Preferably, the linking group has a structure represented by -X¹¹-L¹¹-. Here, X¹¹ represents an oxygen atom or a sulfur atom and is preferably an oxygen atom. L¹¹ represents a linking group, and is preferably a substituted or unsubstituted alkylene group, or a substituted or unsubstituted arylene group, more preferably a substituted or unsubstituted alkylene group having 1 to 10 carbon atoms, or a substituted or unsubstituted phenylene group.

In the formula (11) or (12), R¹⁰¹, R¹⁰², R¹⁰³ and R¹⁰⁴ each independently represent a substituent. Preferably, the substituent is a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom, or a chlorine atom, and even more preferably an unsubstituted alkyl group having 1 to 3 carbon atoms, or an unsubstituted alkoxy group having 1 to 3 carbon atoms.

The linking group represented by L¹ and L² may bond to any of R¹ to R³ in the structure of the formula (1a) or the formula (1b) constituting Q, any of R¹¹ to R²⁰ in the formula (2), any of R²¹ to R²⁴, and R²⁷ to R³⁰ in the formula (3), any of R³¹ to R³⁸ in the formula (4), any of R⁴¹ to R⁴⁸ in the formula (5), any of R⁵¹ to R⁵⁹ in the formula (6), any of R⁵¹ to R⁵⁸, and R⁶¹ to R⁶⁵ in the formula (6′), or any of R⁷¹ to R⁸⁰ in the formula (7). Two or more linking groups may bond to one Q to form a crosslinked structure or a network structure.

Preferably, the structure represented by the formula (11) or (12) is so determined as not to excessively detract from the advantageous effects of the preset invention.

Specific examples of the structure of the recurring unit include structures represented by the following formulae (13) to (16).

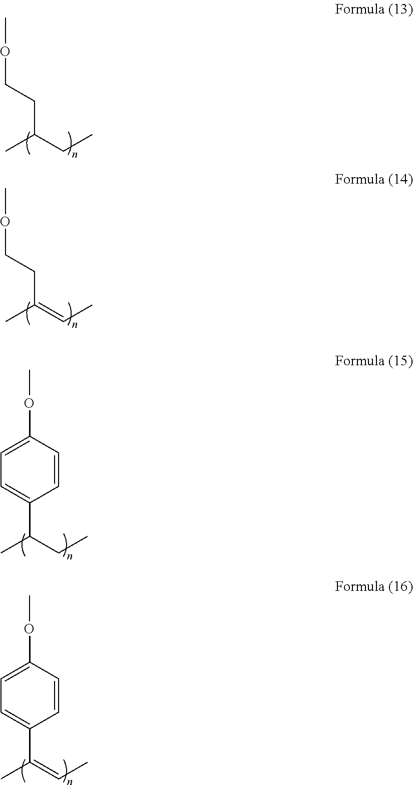

The polymer having the recurring unit containing the structure represented by any of the formulae (13) to (16) may be synthesized in such a manner that a hydroxy group is introduced to any of R¹ to R⁴ in the structure represented by the formula (1a) or any of R¹ to R⁴ and in the formula (b), and the hydroxy group as a linker is reacted with the following compound to introduce a polymerizable group thereinto, followed by polymerizing the polymerizable group.

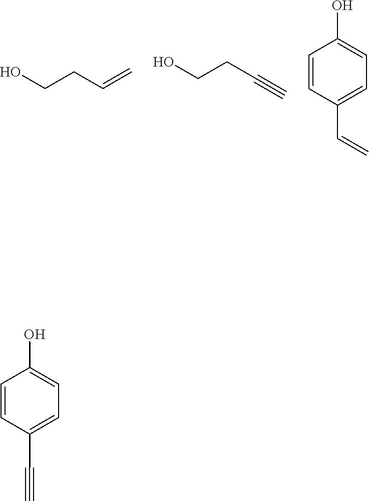

The polymer containing the structure represented by the formula (1a) or the formula (1b) in the molecule may be a polymer containing only a recurring unit having the structure represented by the formula (1a) or the formula (1b), or a polymer further containing a recurring unit having another structure. The recurring unit having the structure represented by the formula (1a) or the formula (1b) contained in the polymer may be only one kind or two or more kinds. Here, two or more kinds include all the case where the recurring unit having the structure represented by the formula (1a) is two or more kinds, the case where the recurring unit having the structure represented by the formula (1b) is two or more kinds, and the case where the recurring unit having the structure represented by the formula (1a) is one or more kinds, and the recurring unit having the structure represented by the formula (1b) is one or more kinds. Examples of the recurring unit that does not have the structure represented by the formula (1a) or the formula (1b) include a recurring unit derived from a monomer that is used for ordinary copolymerization. Examples of the recurring unit include a recurring unit derived from a monomer having an ethylenic unsaturated bond, such as ethylene and styrene.

[Synthesis Method for Compound Represented by Formula (1a) of Formula (1 b)]

The compound represented by the formula (1a) or the formula (1 b) is a novel compound. The compound can be synthesized by combining known reactions.

Specifically, for example, the compound represented by the formula (1a) can be synthesized according to the following scheme.

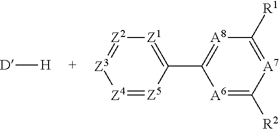

In the above-mentioned scheme, D′ represents a donor group, H represents a hydrogen atom, R¹ and R² each represent an aliphatic group, and at least one of them represents a polycyclic aliphatic group. Z¹ to Z⁵ each independently represent N or C—R^{3′}, and R^{3′} represents a hydrogen atom or a substituent. However, at least one of Z¹ to Z⁵ is C—R^{3′}, and R^{3′} is a halogen atom. Regarding the groups represented by the donor group, and A⁷ to A⁸, R¹ and R², reference may be made to the corresponding description and preferred ranges of those in the formula (a) mentioned above. The halogen atom of R^{3′} includes a fluorine atom, a chlorine atom, a bromine atom and an iodine atom, and is, for example, a bromine atom. Regarding the description of the other substituents than a halogen atom for R^{3′}, reference may be made to the description of the other substituent than a donor group for R³ in the formula (1a).

The compound represented by the formula (1b) can be synthesized according to the above-mentioned scheme but using H-D-H (where D represents a donor-like linking group, and H represents a hydrogen atom) in place of D′-H in the scheme. Regarding the description, the preferred range and specific examples of D, reference may be made to the corresponding description and preferred range thereof in the formula (1b) mentioned above.

For the details of the reaction condition and the reaction process, reference may be made to the corresponding description in the section of Examples given hereinunder.

### [Organic Light Emitting Device]

The compound represented by the formula (1a) or the formula (1b) of the present invention has a light emitting property and can emit an especially good blue light, and is therefore useful as a light emitting material for organic light emitting devices. Using the compound represented by the formula (1a) or the formula (1b) of the present invention, an organic light emitting device capable of emitting a good blue light can be readily realized. For example, there can be provided a compound capable of emitting a light such that the chromaticity coordinate in a color system x is 0.23 or less and y is 0.23 or less, preferably x is 0.20 or less and y is 0.20 or less, more preferably x is 0.15 or less and y is 0.20 or less, even more preferably x is 0.15 or less and y is 0.15 or less, especially more preferably x is 0.15 or less and y is 0.13 or less. Consequently, the compound represented by the formula (1a) or the formula (1b) of the present invention can be effectively used as a light emitting material in a light emitting layer of an organic light emitting device.

The compound represented by the formula (1a) or the formula (1b) include a delayed fluorescent material (delayed phosphor) that emits delayed fluorescence. Specifically, the present invention includes an invention of a delayed fluorescent material having a structure represented by the formula (1a) or the formula (1b), an invention of using the compound represented by the formula (1a) or the formula (1b) as a delayed fluorescent material, and an invention of a method of using the compound represented by the formula (1a) or the formula (1b) for emitting delayed fluorescence. An organic light emitting device using such a compound as a light emitting material is characterized that it emits delayed fluorescence and has a high emission efficiency. The principle will be described below with reference to an organic electroluminescent device taken as an example.

In an organic electroluminescent device, carriers are injected from an anode and a cathode to a light emitting material to form an excited state for the light emitting material, with which light is emitted. In the case of a carrier injection type organic electroluminescent device, in general, excitons that are excited to the excited singlet state are 25% of the total excitons generated, and the remaining 75% thereof are excited to the excited triplet state. Accordingly, the use of phosphorescence, which is light emission from the excited triplet state, provides a high energy utilization. However, the excited triplet state has a long lifetime and thus causes saturation of the excited state and deactivation of energy through mutual action with the excitons in the excited triplet state, and therefore the quantum yield of phosphorescence may generally be often not high. A delayed fluorescent material emits fluorescent light through the mechanism that the energy of excitons transits to the excited triplet state through intersystem crossing or the like, and then transits to the excited singlet state through reverse intersystem crossing due to triplet-triplet annihilation or absorption of thermal energy, thereby emitting fluorescent light. It is considered that among the materials, a thermal activation type delayed fluorescent material emitting light through absorption of thermal energy is particularly useful for an organic electroluminescent device. In the case where a delayed fluorescent material is used in an organic electroluminescent device, the excitons in the excited singlet state normally emit fluorescent light. On the other hand, the excitons in the excited triplet state emit fluorescent light through intersystem crossing to the excited singlet state by absorbing the heat generated by the device. At this time, the light emitted through reverse intersystem crossing from the excited triplet state to the excited singlet state has the same wavelength as fluorescent light since it is light emission from the excited singlet state, but has a longer lifetime (light emission lifetime) than the normal fluorescent light, and thus the light is observed as fluorescent light that is delayed from the normal fluorescent light. The light may be defined as delayed fluorescent light. The use of the thermal activation type exciton transition mechanism may raise the proportion of the compound in the excited singlet state, which is generally formed in a proportion only of 25%, to 25% or more through the absorption of the thermal energy after the carrier injection. A compound that emits strong fluorescent light and delayed fluorescent light at a low temperature of lower than 100° C. undergoes the intersystem crossing from the excited triplet state to the excited singlet state sufficiently with the heat of the device, thereby emitting delayed fluorescent light, and thus the use of the compound may drastically enhance the light emission efficiency.

Further, the compound represented by the formula (1a) or the formula (1b) of the present invention can form a film according to a vapor evaporation method, and in addition, can be readily dissolved in a solvent and can form a film according to a coating method. In any case of using such a film formation method, the compound expresses the above-mentioned excellent light emitting characteristics. In addition, in the case where a coating method is employed as a film formation method using the compound represented by the formula (1a) or the formula (1b), an organic film of the compound can be efficiently formed in a uniform film thickness without using any large scale film formation apparatus, and therefore the compound of the present invention can significantly enhance the production efficiency of organic light emitting devices. Further, the compound represented by the formula (1a) or the formula (1b) has high thermal stability and is therefore excellent in practical use. Consequently, the organic light emitting device containing the compound can realize stable light emission performance even in high-temperature environments, and therefore can be effectively used, for example, for car navigation systems, display devices of head-up displays, in-vehicle illuminations and outdoor displays.

Using the compound represented by the formula (a) or the formula (1b) of the present invention as a light emitting material in a light emitting layer, excellent organic light emitting devices such as an organic photoluminescent device (organic PL device) and an organic electroluminescent device (organic EL device) can be provided. In these, the compound represented by the formula (1a) or the formula (1b) of the present invention can have a function of assisting light emission of any other light emitting material contained in a light emitting layer, as a so-called assist dopant. Specifically, the compound represented by the formula (1a) or the formula (1b) of the present invention contained in a light emitting layer may have a lowest excited singlet energy level between the lowest excited singlet energy level of the host material contained in the light emitting layer and the lowest excited singlet energy level of the other light emitting material contained in the light emitting layer.

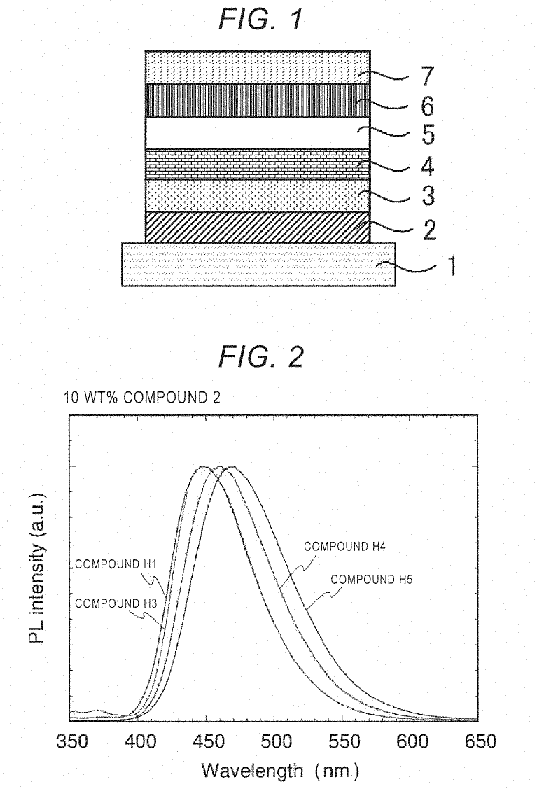

An organic photoluminescent device has a structure where at least a light emitting layer is formed on a substrate. An organic electroluminescent device has a structure including at least an anode, a cathode and an organic layer formed between the anode and the cathode. The organic layer contains at least a light emitting layer, and may be formed of a light emitting layer alone, or may has one or more other organic layers in addition to a light emitting layer. The other organic layers include a hole transport layer, a hole injection layer, an electron blocking layer, a hole blocking layer, an electron injection layer, an electron transport layer, and an exciton blocking layer. The hole transport layer may be a hole injection transport layer having a hole injection function, and the electron transport layer may be an electron injection transport layer having an electron injection function. A configuration example of an organic electroluminescent device is shown in FIG. 1. In FIG. 1, 1 is a substrate, 2 is an anode, 3 is a hole injection layer, 4 is a hole transport layer, 5 is a light emitting layer, 6 is an electron transport layer, and 7 is a cathode.

In the following, the constituent members and the layers of the organic electroluminescent device are described. The description of the substrate and the light emitting layer given below may apply to the substrate and the light emitting layer of an organic photoluminescent device.

### (Substrate)

The organic electroluminescent device of the invention is preferably supported by a substrate. The substrate is not particularly limited and may be those that have been commonly used in an organic electroluminescent device, and examples thereof used include those formed of glass, transparent plastics, quartz and silicon.

### (Anode)

The anode of the organic electroluminescent device used is preferably formed of, as an electrode material, a metal, an alloy, or an electroconductive compound each having a large work function (4 eV or more), or a mixture thereof. Specific examples of the electrode material include a metal, such as Au, and an electroconductive transparent material, such as CuI, indium tin oxide (ITO), SnO₂ and ZnO. A material that is amorphous and is capable of forming a transparent electroconductive film, such as IDIXO (In₂O₃—ZnO), may also be used. The anode may be formed in such a manner that the electrode material is formed into a thin film by such a method as vapor deposition or sputtering, and the film is patterned into a desired pattern by a photolithography method, or in the case where the pattern may not require high accuracy (for example, approximately 100 μm or more), the pattern may be formed with a mask having a desired shape on vapor deposition or sputtering of the electrode material. In alternative, in the case where a material capable of being coated, such as an organic electroconductive compound, is used, a wet film forming method, such as a printing method and a coating method, may be used. In the case where emitted light is to be taken out through the anode, the anode preferably has a transmittance of more than 10%, and the anode preferably has a sheet resistance of several hundred Ω/sq (ohm per square) or less. The thickness of the anode may be generally selected from a range of from 10 to 1,000 nm, and preferably from 10 to 200 nm, while depending on the material used.

### (Cathode)

The cathode is preferably formed of as an electrode material a metal (which is referred to as an electron injection metal), an alloy, or an electroconductive compound, having a small work function (4 eV or less), or a mixture thereof. Specific examples of the electrode material include sodium, a sodium-potassium alloy, magnesium, lithium, a magnesium-cupper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth metal. Among these, a mixture of an electron injection metal and a second metal that is a stable metal having a larger work function than the electron injection metal, for example, a magnesium-silver mixture, a magnesium-aluminum mixture, a magnesium-indium mixture, an aluminum-aluminum oxide (Al₂O₃) mixture, a lithium-aluminum mixture, and aluminum, is preferred from the standpoint of the electron injection property and the durability against oxidation and the like. The cathode may be produced by forming the electrode material into a thin film by such a method as vapor deposition or sputtering. The cathode preferably has a sheet resistance of several hundred f/sq (ohm per square) or less, and the thickness thereof may be generally selected from a range of from 10 nm to 5 μm, and preferably from 50 to 200 nm. For transmitting the emitted light, any one of the anode and the cathode of the organic electroluminescent device is preferably transparent or translucent, thereby enhancing the light emission luminance.

The cathode may be formed with the electroconductive transparent materials described for the anode, thereby forming a transparent or translucent cathode, and by applying the cathode, a device having an anode and a cathode, both of which have transmittance, may be produced.

### (Light Emitting Layer)

The light emitting layer is a layer in which holes and electrons injected from an anode and a cathode are recombined to give excitons for light emission. The layer contains a light emitting material and a host material. However, a light emitting alone may be used as the light emitting layer. As the light emitting material, one or more selected from a group of the compounds of the present invention represented by the formula (1a) or the formula (1b) can be used. In order that the organic electroluminescent device and the organic photoluminescent device of the present invention can express a high light emission efficiency, it is important to confine the singlet exciton and the triplet exciton formed in the light emitting material to the light emitting material. Accordingly, in the case where a host material is used, an organic compound, of which at least any one of the excited singlet energy and the excited triplet energy is higher than that of the light emitting material of the present invention, may be used as the host material. As a result, the singlet exciton and the triplet exciton formed in the light emitting material of the present invention can be confined to the molecule of the light emitting material of the present invention to sufficiently derive the light emission efficiency thereof. Needless-to-say, there may be a case where a high light emission efficiency could be attained even though the singlet exciton and the triplet exciton could not be sufficiently confined, and therefore, any host material capable of realizing a high light emission efficiency can be used in the present invention with no specific limitation. In the organic light emitting device or the organic electroluminescent device of the present invention, light emission occurs from the light emitting material of the present invention contained in the light emitting layer. The light emission contains both of fluorescent emission and delayed fluorescent emission. In addition, a part of light emission may be partially from a host material.

In the case where a host material is used, the content of the compound serving as a light emitting material of the present invention in the light emitting layer may be 0.1 to 99.9 wt %. The upper limit may be, for example, less than 50 wt %, or 20 wt % or less, or 10 wt % or less.

The host material in the light emitting layer is preferably an organic compound having hole transport competence and electron transport competence, capable of preventing prolongation of emission wavelength and having a high glass transition temperature.

### (Injection Layer)

The injection layer is a layer that is provided between the electrode and the organic layer, for decreasing the driving voltage and enhancing the light emission luminance, and includes a hole injection layer and an electron injection layer, which may be provided between the anode and the light emitting layer or the hole transport layer and between the cathode and the light emitting layer or the electron transport layer. The injection layer may be provided depending on necessity.

### (Blocking Layer)

The blocking layer is a layer that is capable of inhibiting charges (electrons or holes) and/or excitons present in the light emitting layer from being diffused outside the light emitting layer. The electron blocking layer may be disposed between the light emitting layer and the hole transport layer, and inhibits electrons from passing through the light emitting layer toward the hole transport layer. Similarly, the hole blocking layer may be disposed between the light emitting layer and the electron transport layer, and inhibits holes from passing through the light emitting layer toward the electron transport layer. The blocking layer may also be used for inhibiting excitons from being diffused outside the light emitting layer. Thus, the electron blocking layer and the hole blocking layer each may also have a function as an exciton blocking layer. The term “the electron blocking layer” or “the exciton blocking layer” referred to herein is intended to include a layer that has both the functions of an electron blocking layer and an exciton blocking layer by one layer.

### (Hole Blocking Layer)

The hole blocking layer has the function of an electron transport layer in a broad sense. The hole blocking layer has a function of inhibiting holes from reaching the electron transport layer while transporting electrons, and thereby enhances the recombination probability of electrons and holes in the light emitting layer. As the material for the hole blocking layer, the material for the electron transport layer to be mentioned below may be used optionally.

### (Electron Blocking Layer)

The electron blocking layer has the function of transporting holes in a broad sense. The electron blocking layer has a function of inhibiting electrons from reaching the hole transport layer while transporting holes, and thereby enhances the recombination probability of electrons and holes in the light emitting layer.

### (Exciton Blocking Layer)

The exciton blocking layer is a layer for inhibiting excitons generated through recombination of holes and electrons in the light emitting layer from being diffused to the charge transporting layer, and the use of the layer inserted enables effective confinement of excitons in the light emitting layer, and thereby enhances the light emission efficiency of the device. The exciton blocking layer may be inserted adjacent to the light emitting layer on any of the side of the anode and the side of the cathode, and on both the sides. Specifically, in the case where the exciton blocking layer is present on the side of the anode, the layer may be inserted between the hole transport layer and the light emitting layer and adjacent to the light emitting layer, and in the case where the layer is inserted on the side of the cathode, the layer may be inserted between the light emitting layer and the cathode and adjacent to the light emitting layer. Between the anode and the exciton blocking layer that is adjacent to the light emitting layer on the side of the anode, a hole injection layer, an electron blocking layer and the like may be provided, and between the cathode and the exciton blocking layer that is adjacent to the light emitting layer on the side of the cathode, an electron injection layer, an electron transport layer, a hole blocking layer and the like may be provided. In the case where the blocking layer is provided, preferably, at least one of the excited singlet energy and the excited triplet energy of the material used as the blocking layer is higher than the excited singlet energy and the excited triplet energy of the light emitting layer, respectively, of the light emitting material.

### (Hole Transport Layer)

The hole transport layer is formed of a hole transport material having a function of transporting holes, and the hole transport layer may be provided as a single layer or plural layers.

The hole transport material has one of injection or transporting property of holes and blocking property of electrons, and may be any of an organic material and an inorganic material. Examples of known hole transport materials that may be used herein include a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer, particularly a thiophene oligomer. Among these, a porphyrin compound, an aromatic tertiary amine compound and a styrylamine compound are preferably used, and an aromatic tertiary amine compound is more preferably used.

### (Electron Transport Layer)

The electron transport layer is formed of a material having a function of transporting electrons, and the electron transport layer may be a single layer or may be formed of plural layers.

The electron transport material (often also acting as a hole blocking material) may have a function of transmitting the electrons injected from a cathode to a light emitting layer. The electron transport layer usable here includes, for example, nitro-substituted fluorene derivatives, diphenylquinone derivatives, thiopyran dioxide derivatives, carbodiimides, fluorenylidenemethane derivatives, anthraquinodimethane and anthrone derivatives, oxadiazole derivatives, etc. Further, thiadiazole derivatives derived from the above-mentioned oxadiazole derivatives by substituting the oxygen atom in the oxadiazole ring with a sulfur atom, and quinoxaline derivatives having a quinoxaline ring known as an electron-attractive group are also usable as the electron transport material. Further, polymer materials prepared by introducing these materials into the polymer chain, or having these material in the polymer main chain are also usable.

In producing the organic electroluminescent device, the compound represented by the formula (1a) or the formula (1b) may be used not only in the light emitting layer but also in any other layer than the light emitting layer. In so doing, the compound represented by the formula (1a) or the formula (1b) used in the light emitting layer and the compound represented by the formula (1a) or the formula (1b) used in the other layer than the light emitting layer may be the same as or different from each other. For example, the compound represented by the formula (1a) or the formula (1b) may be used in the above-mentioned injection layer, the blocking layer, the hole blocking layer, the electron blocking layer, the exciton blocking layer, the hole transport layer, and the electron transport layer. The method for forming these layers is not specifically limited, and the layers may be formed according to any of a dry process or a wet process.

Preferred materials for use for the organic electroluminescent device are concretely exemplified below. However, the materials for use in the present invention are not limitatively interpreted by the following exemplary compounds. Compounds, even though exemplified as materials having a specific function, can also be used as other materials having any other function. In the structural formulae of the following exemplary compounds, R, and R₁ to R₁₀ each independently represent a hydrogen atom or a substituent. n represents an integer of 3 to 5.

First, preferred compounds for use as a host material in a light emitting layer are mentioned below.

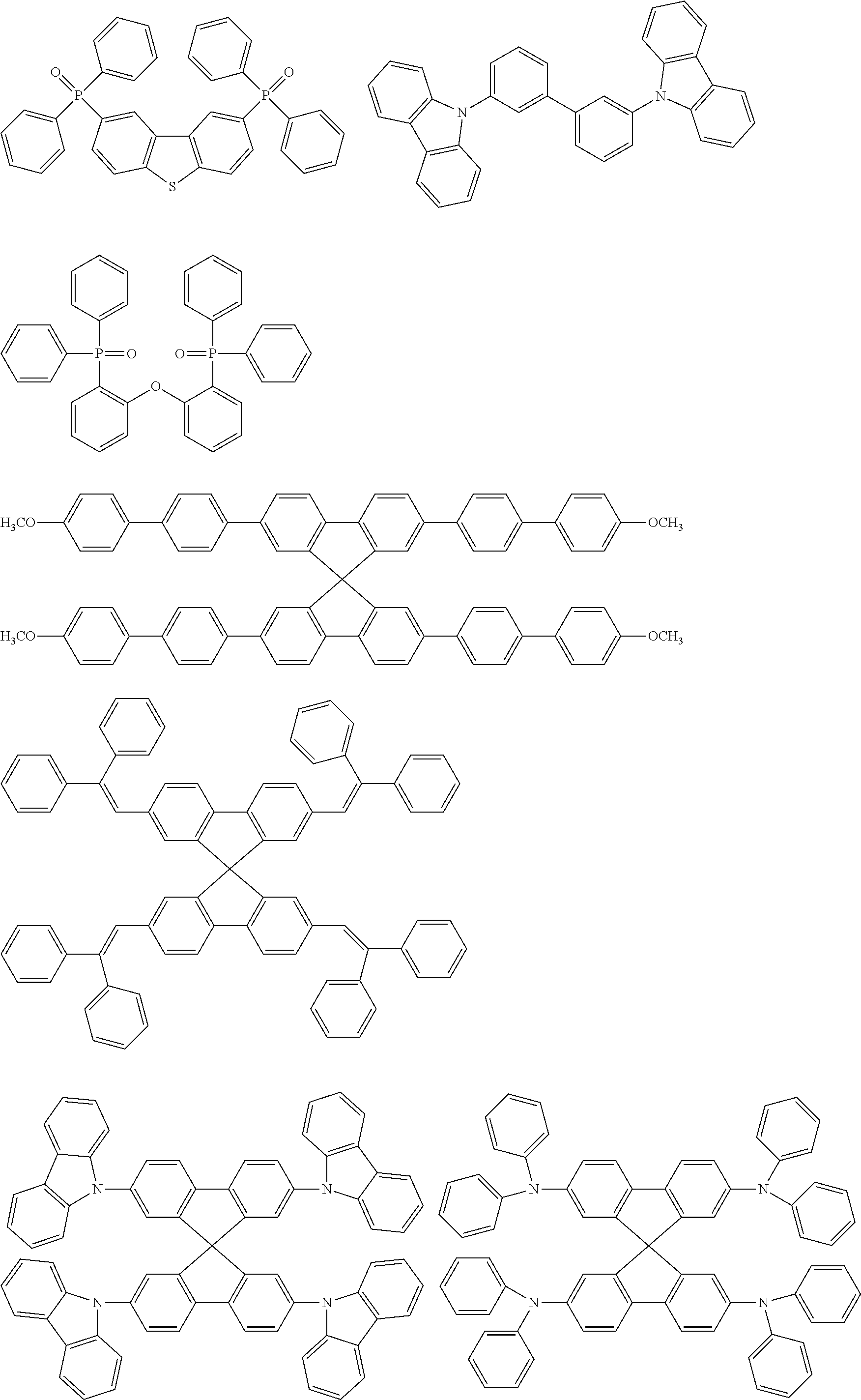

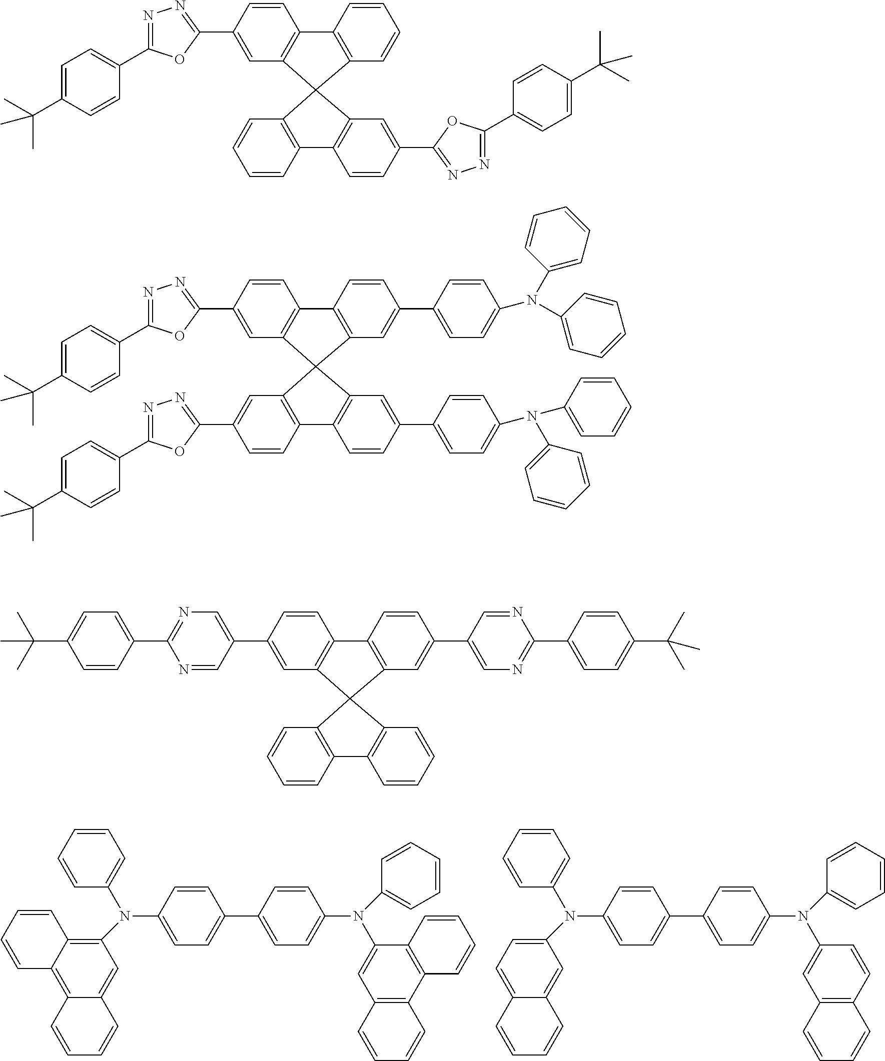

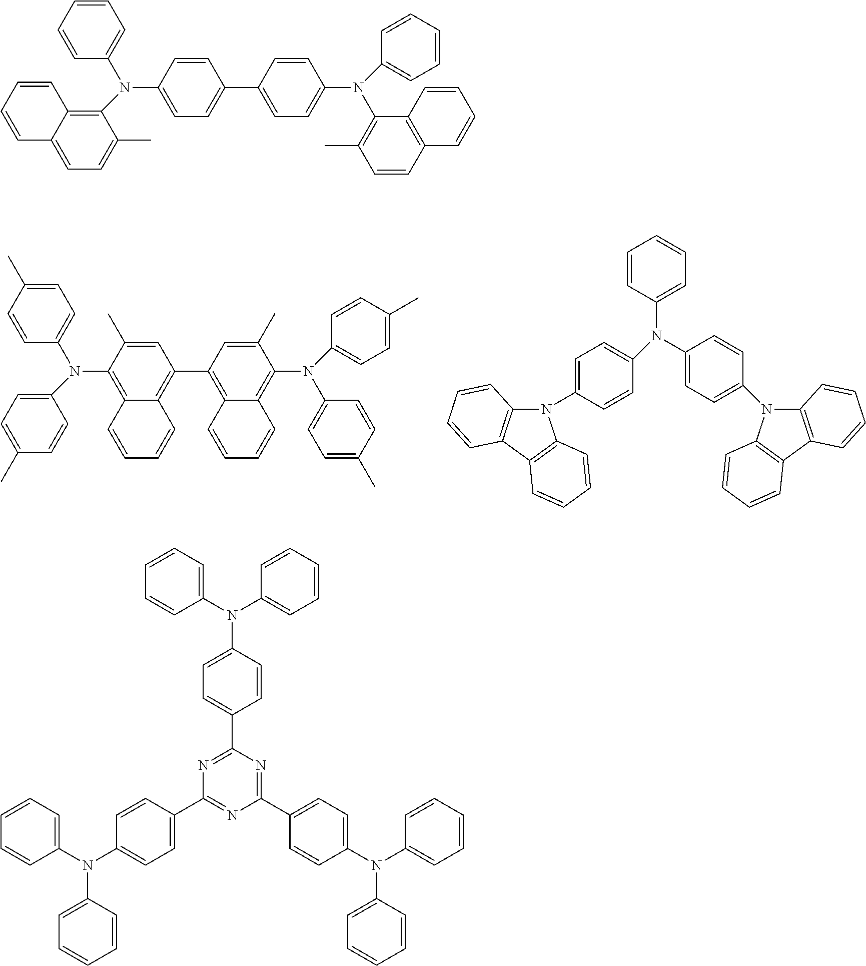 [SMILES: Cc1ccc2ccccc2c1N(c1ccccc1)c1ccc(-c2ccc(N(c3ccccc3)c3c(C)ccc4ccccc34)cc2)cc1 ; Cc1ccc(N(c2ccc(C)cc2)c2c(C)cc(-c3cc(C)c(N(c4ccc(C)cc4)c4ccc(C)cc4)c4ccccc34)c3ccccc23)cc1 ; c1ccc(N(c2ccc(-n3c4ccccc4c4ccccc43)cc2)c2ccc(-n3c4ccccc4c4ccccc43)cc2)cc1 ; c1ccc(N(c2ccccc2)c2ccc(-c3nc(-c4ccc(N(c5ccccc5)c5ccccc5)cc4)nc(-c4ccc(N(c5ccccc5)c5ccccc5)cc4)n3)cc2)cc1]

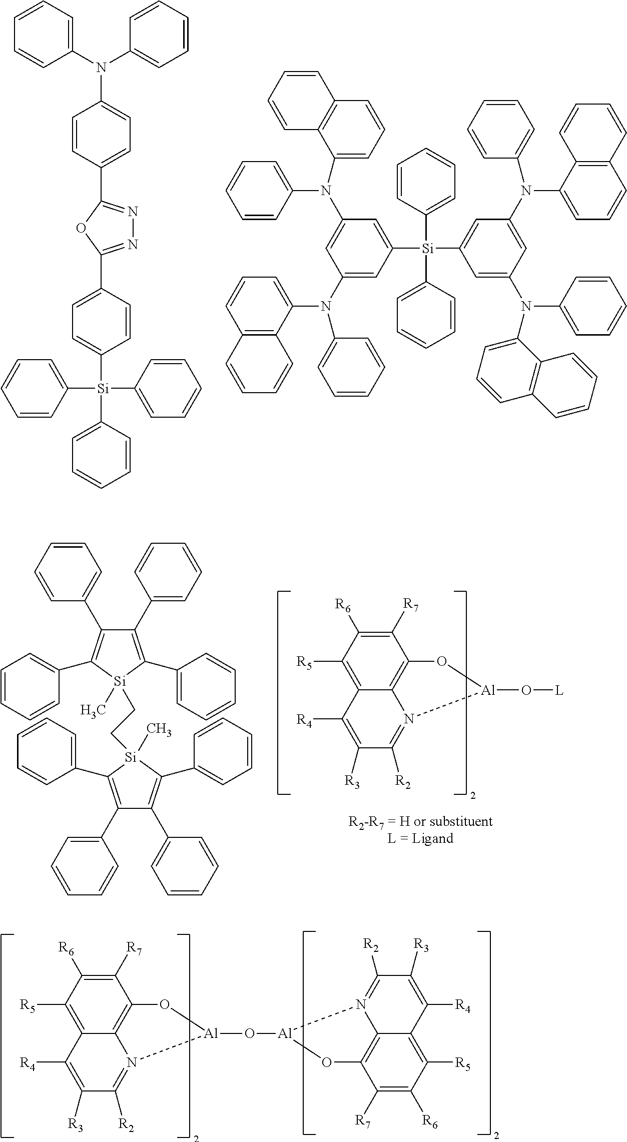

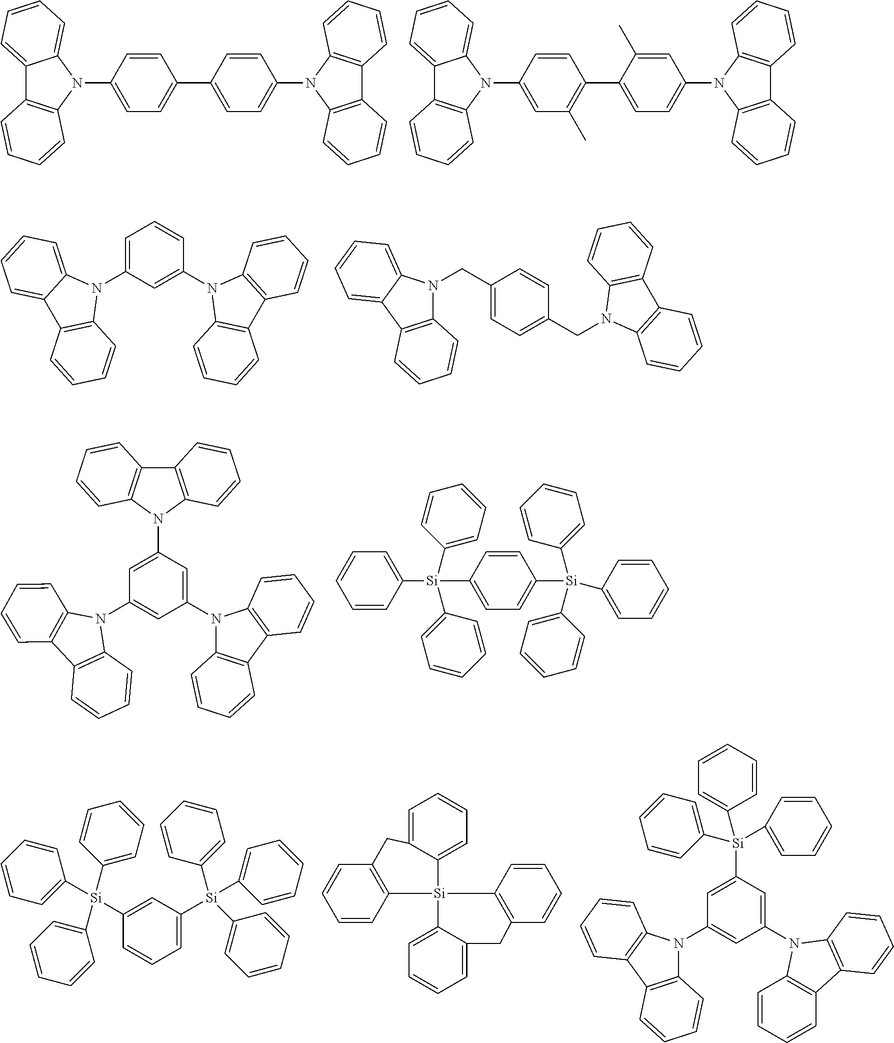

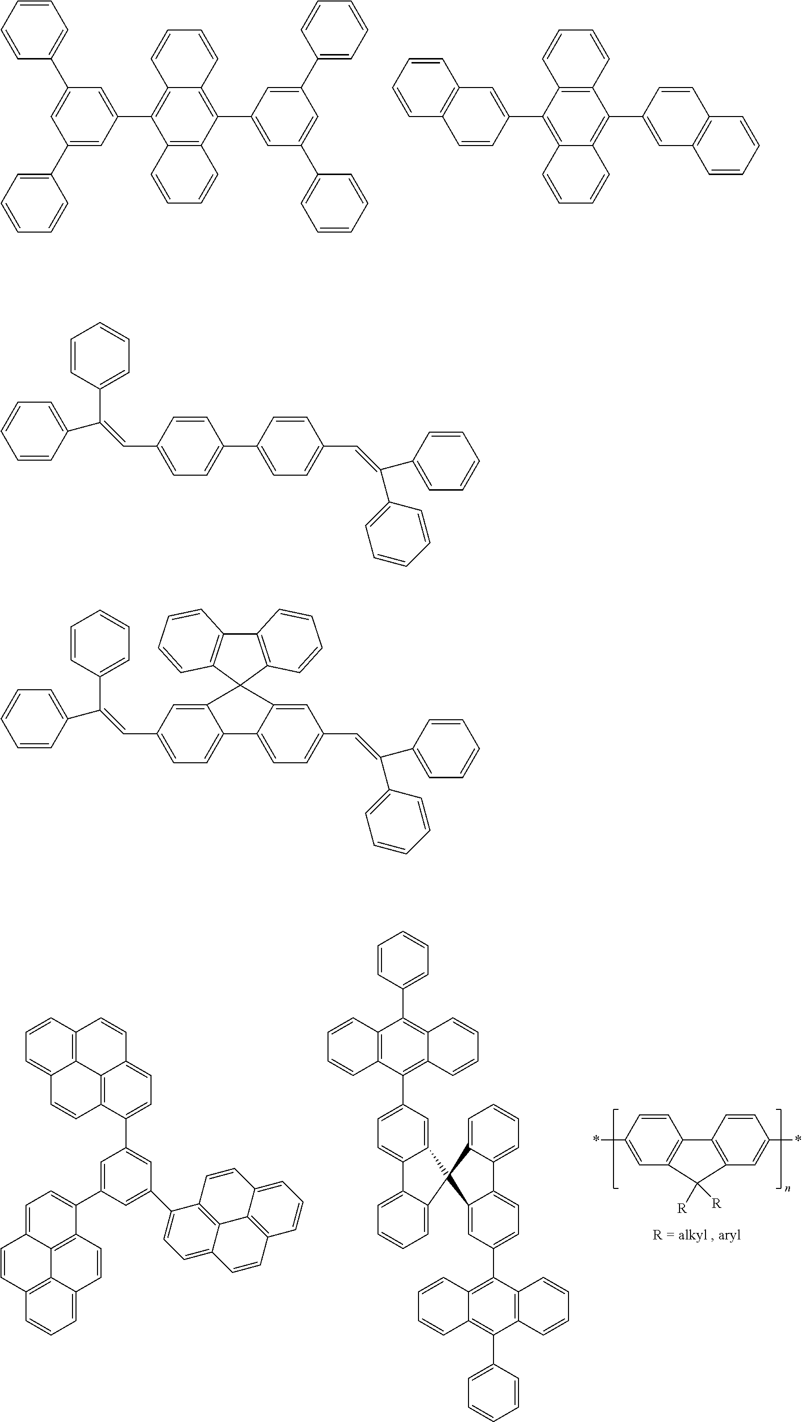

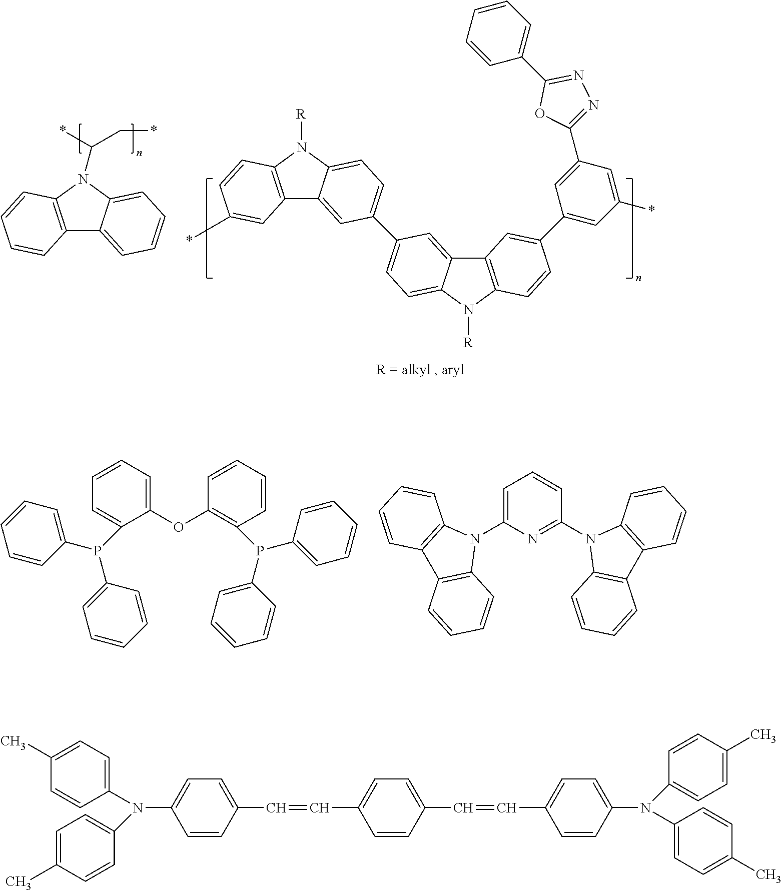

Next, preferred compounds for use as a hole injection material are mentioned below.

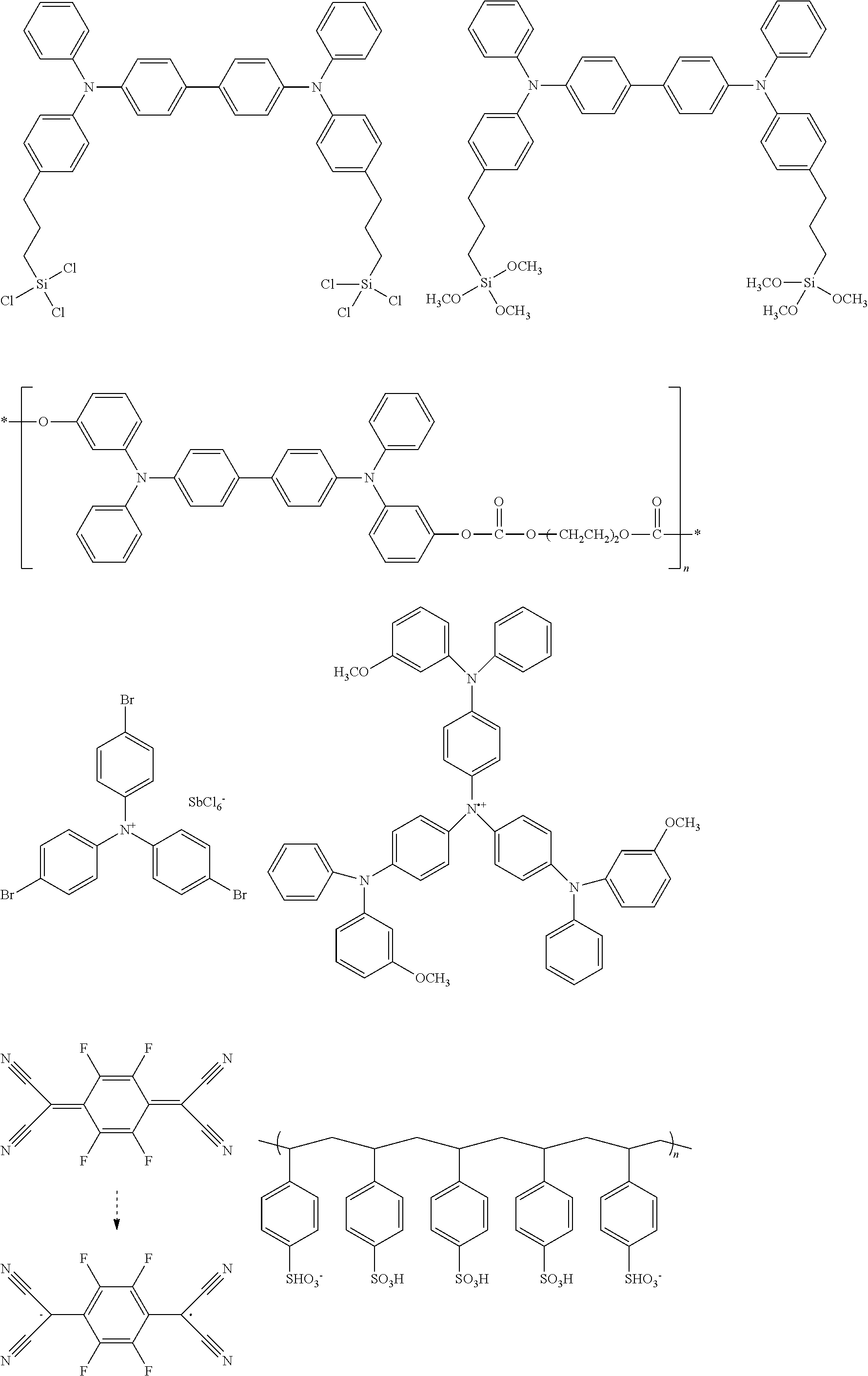

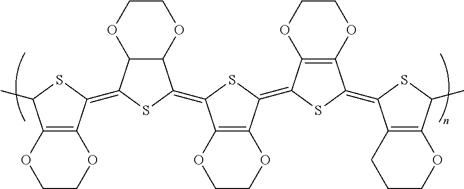

Next, preferred compounds for use as a hole transport material are mentioned below.

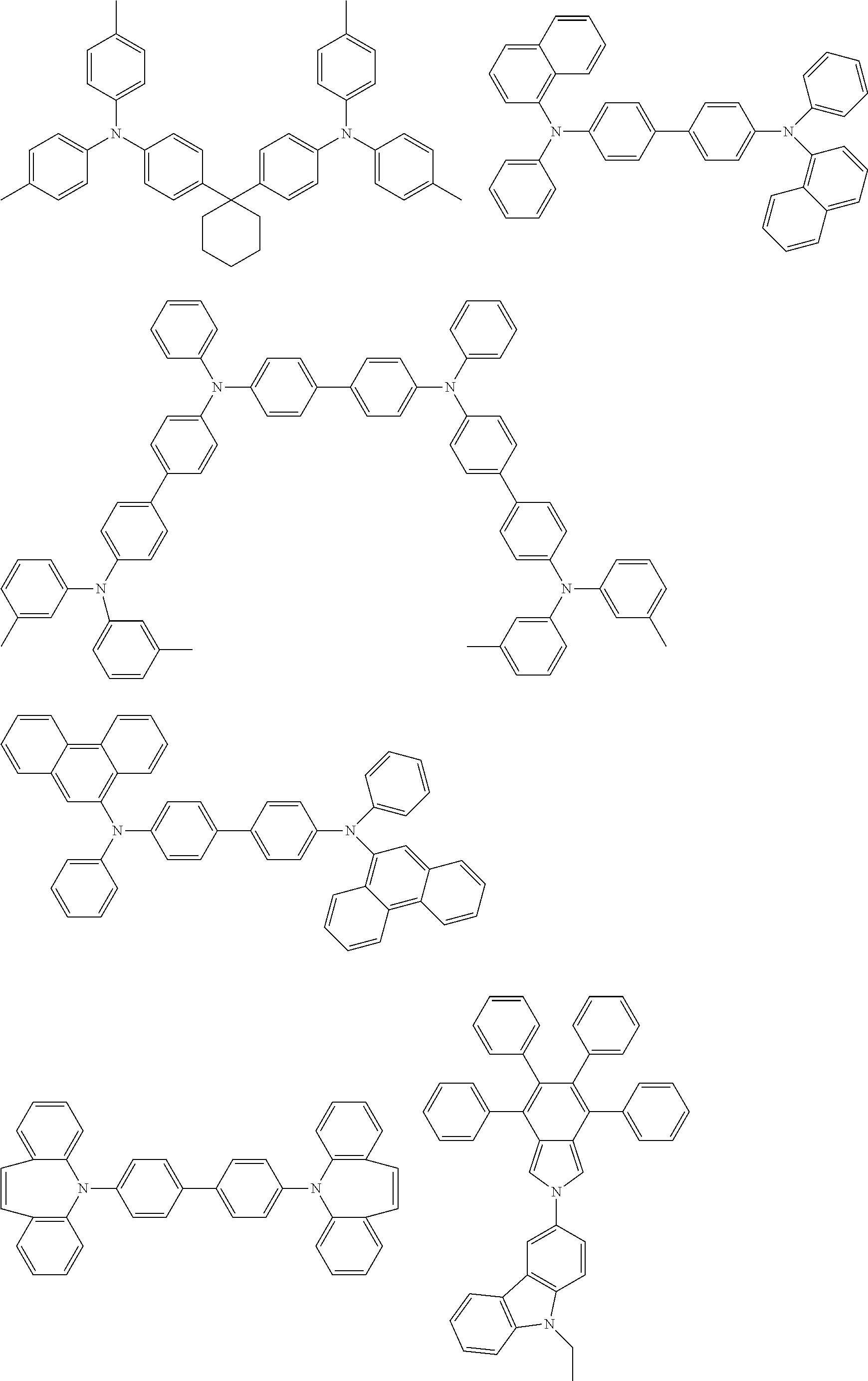

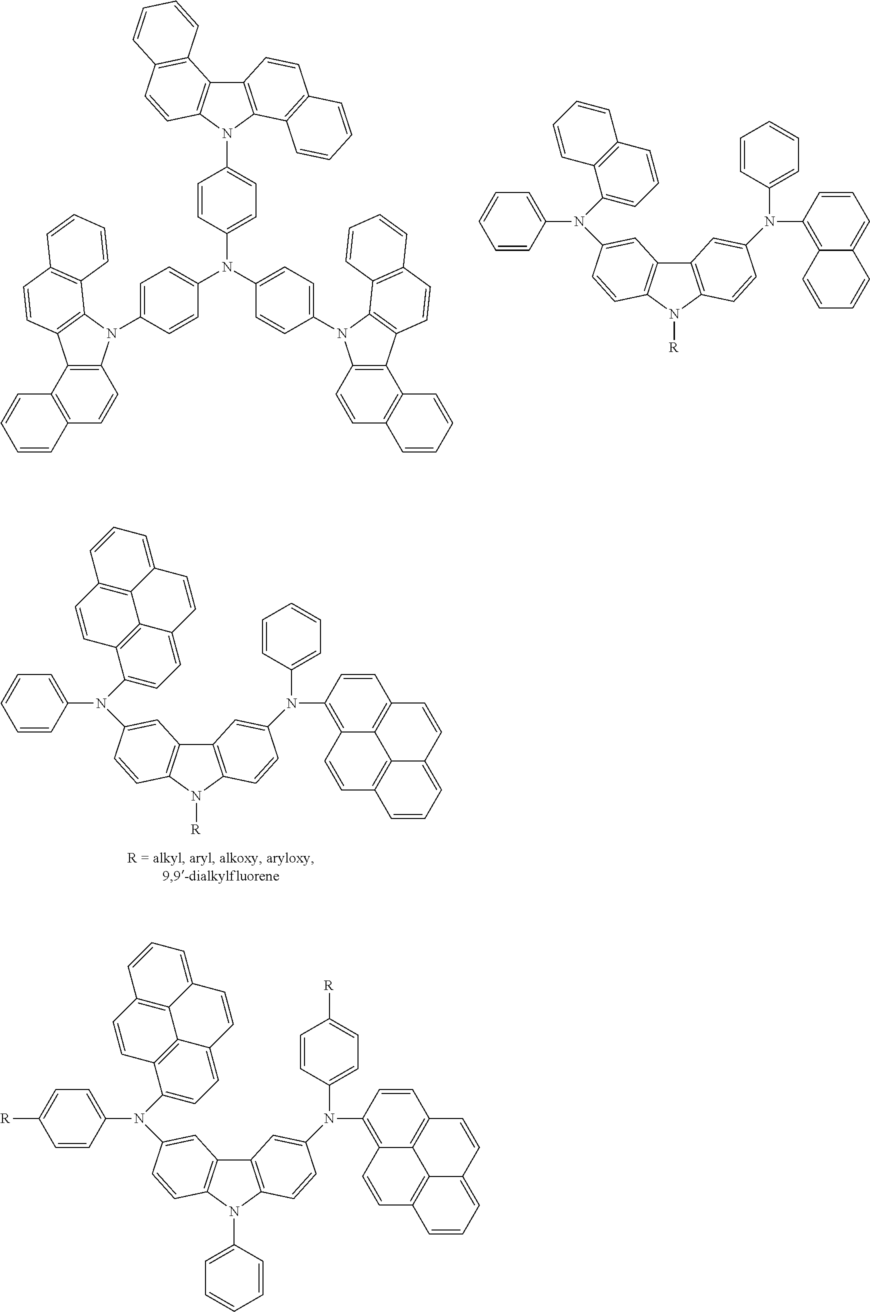

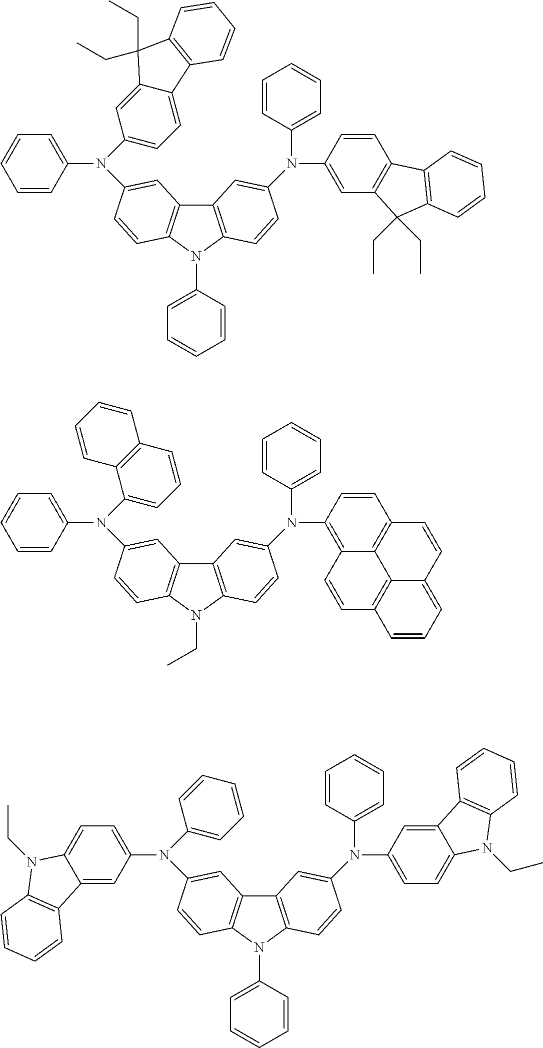 [SMILES: CCC1(CC)c2ccccc2-c2ccc(N(c3ccccc3)c3ccc4c(c3)c3cc(N(c5ccccc5)c5ccc6c(c5)C(CC)(CC)c5ccccc5-6)ccc3n4-c3ccccc3)cc21 ; CCn1c2ccc(N(c3ccccc3)c3cccc4ccccc34)cc2c2cc(N(c3ccccc3)c3ccc4ccc5cccc6ccc3c4c56)ccc21 ; CCn1c2ccccc2c2cc(N(c3ccccc3)c3ccc4c(c3)c3cc(N(c5ccccc5)c5ccc6c(c5)c5ccccc5n6CC)ccc3n4-c3ccccc3)ccc21]

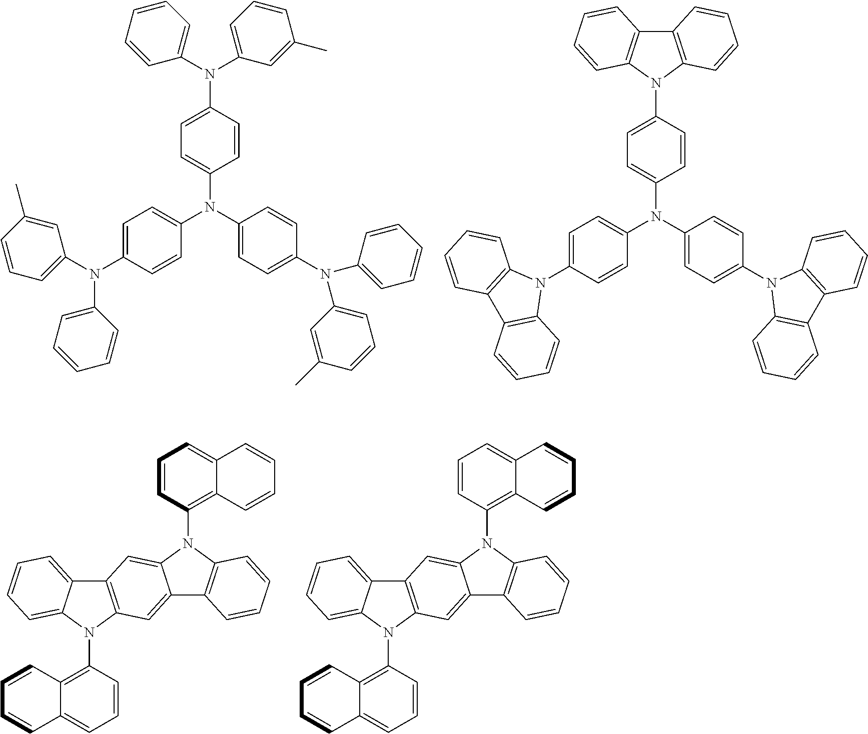

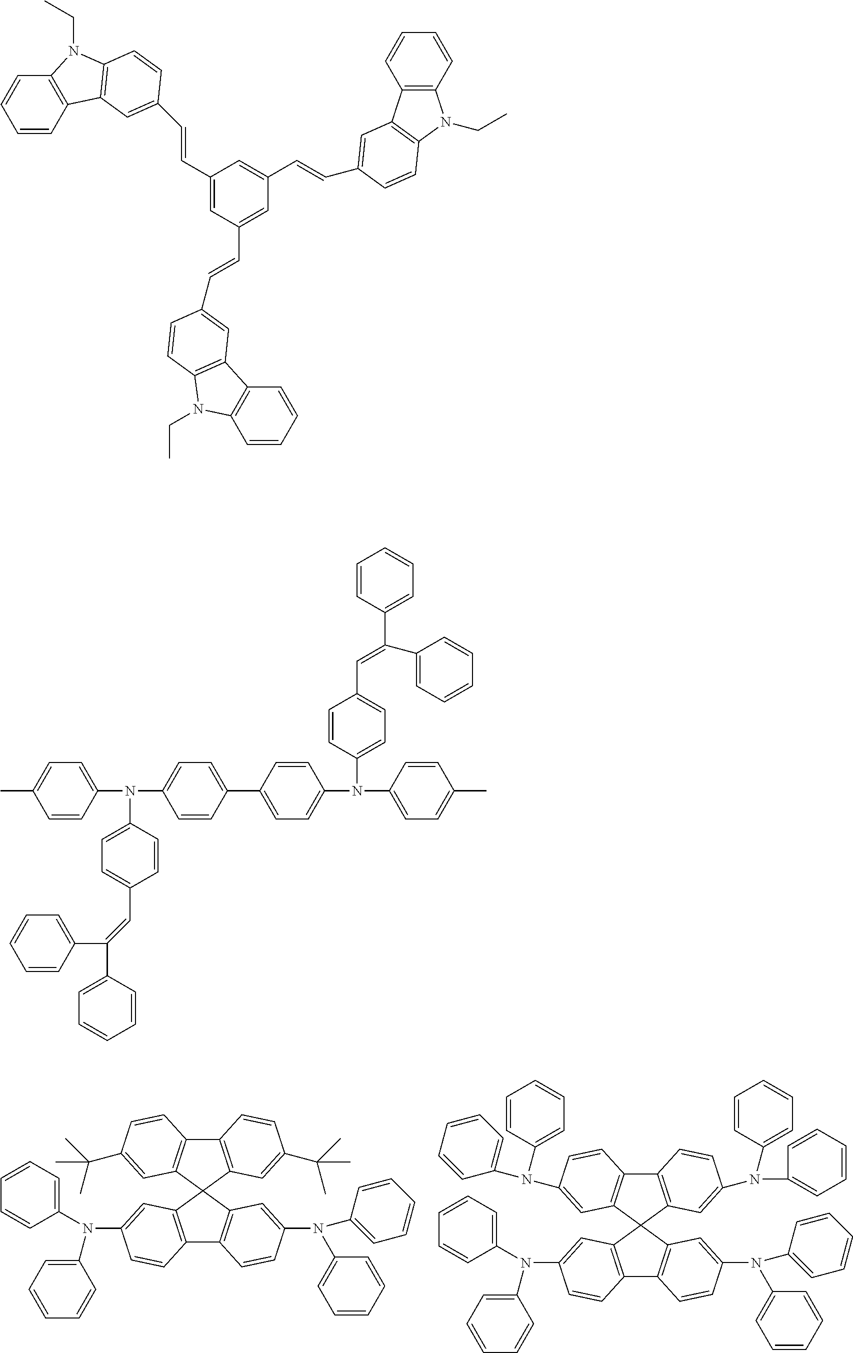

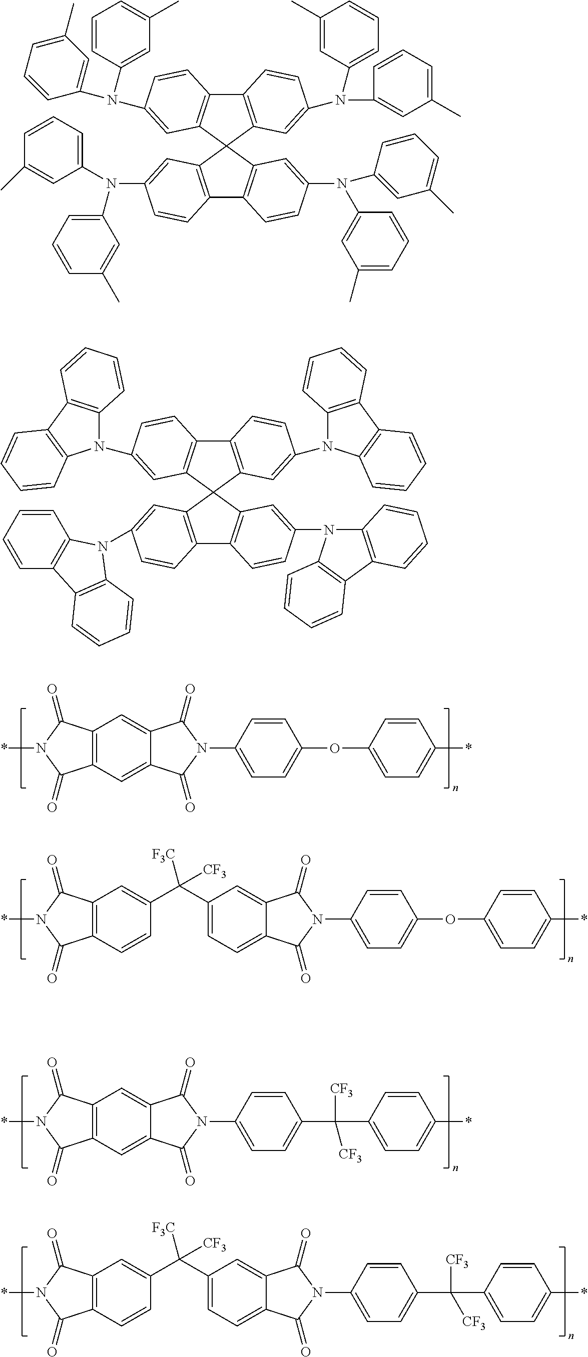

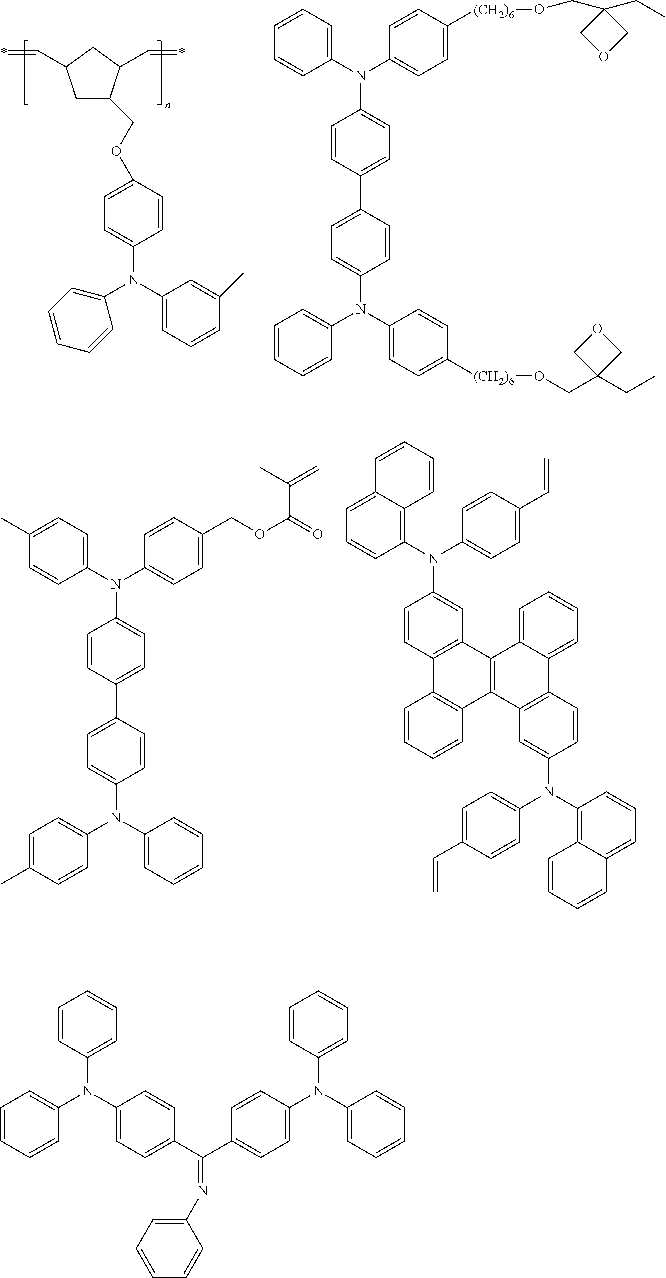

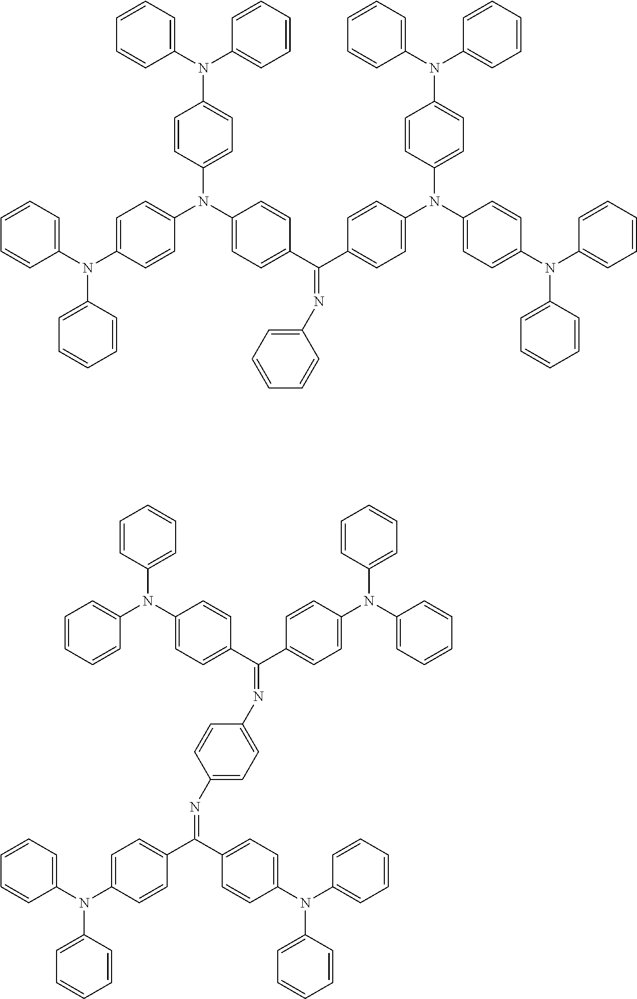 [SMILES: c1ccc(N=C(c2ccc(N(c3ccc(N(c4ccccc4)c4ccccc4)cc3)c3ccc(N(c4ccccc4)c4ccccc4)cc3)cc2)c2ccc(N(c3ccc(N(c4ccccc4)c4ccccc4)cc3)c3ccc(N(c4ccccc4)c4ccccc4)cc3)cc2)cc1 ; c1ccc(N(c2ccccc2)c2ccc(C(=Nc3ccc(N=C(c4ccc(N(c5ccccc5)c5ccccc5)cc4)c4ccc(N(c5ccccc5)c5ccccc5)cc4)cc3)c3ccc(N(c4ccccc4)c4ccccc4)cc3)cc2)cc1]

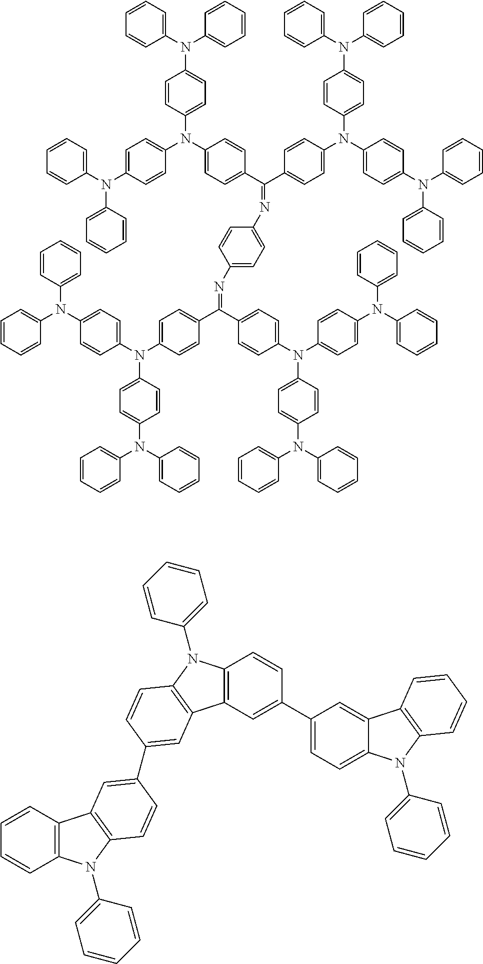 [SMILES: c1ccc(N(c2ccccc2)c2ccc(N(c3ccc(C(=Nc4ccc(N=C(c5ccc(N(c6ccc(N(c7ccccc7)c7ccccc7)cc6)c6ccc(N(c7ccccc7)c7ccccc7)cc6)cc5)c5ccc(N(c6ccc(N(c7ccccc7)c7ccccc7)cc6)c6ccc(N(c7ccccc7)c7ccccc7)cc6)cc5)cc4)c4ccc(N(c5ccc(N(c6ccccc6)c6ccccc6)cc5)c5ccc(N(c6ccccc6)c6ccccc6)cc5)cc4)cc3)c3ccc(N(c4ccccc4)c4ccccc4)cc3)cc2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

Next, preferred compounds for use as an electron blocking material are mentioned below.

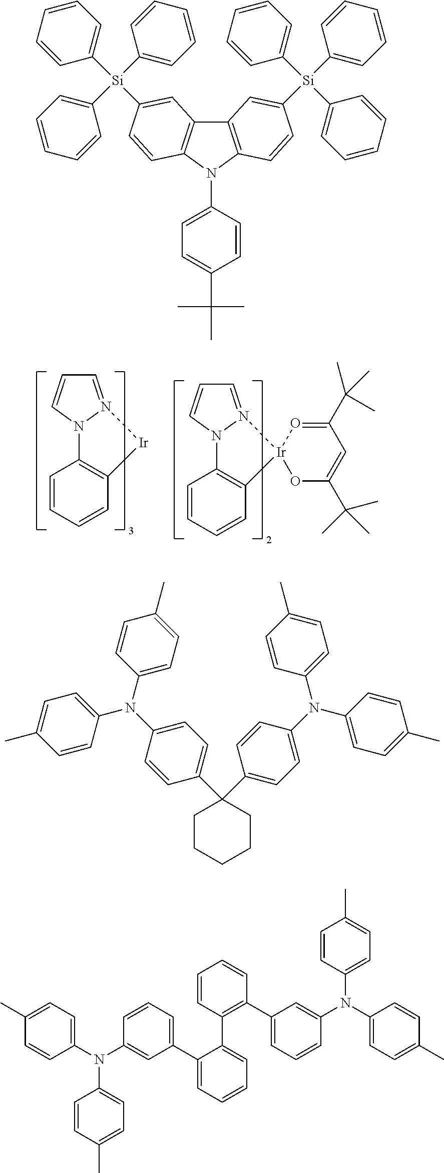

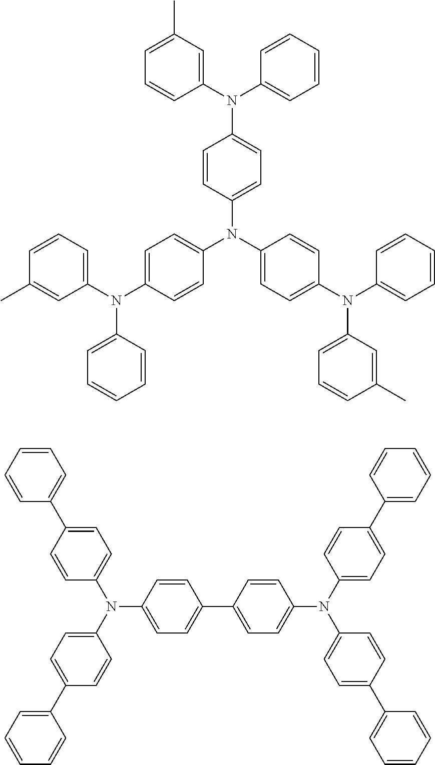 [SMILES: Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

Next, preferred compounds for use as a hole blocking material are mentioned below.

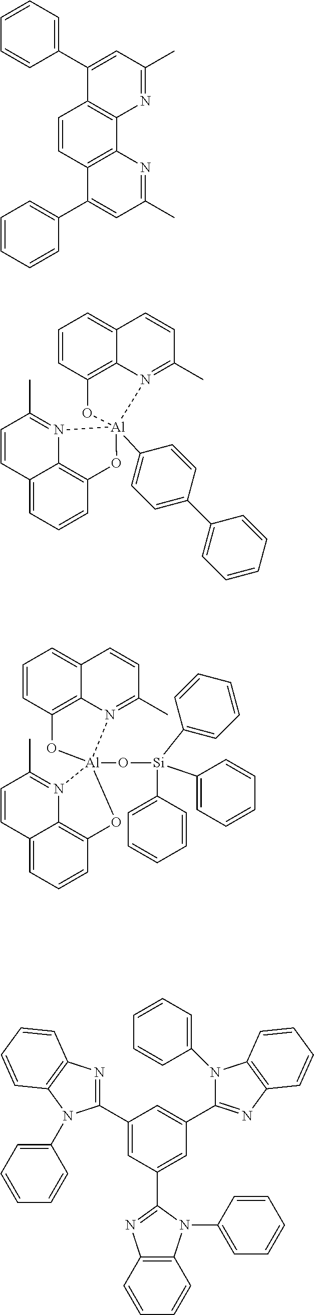

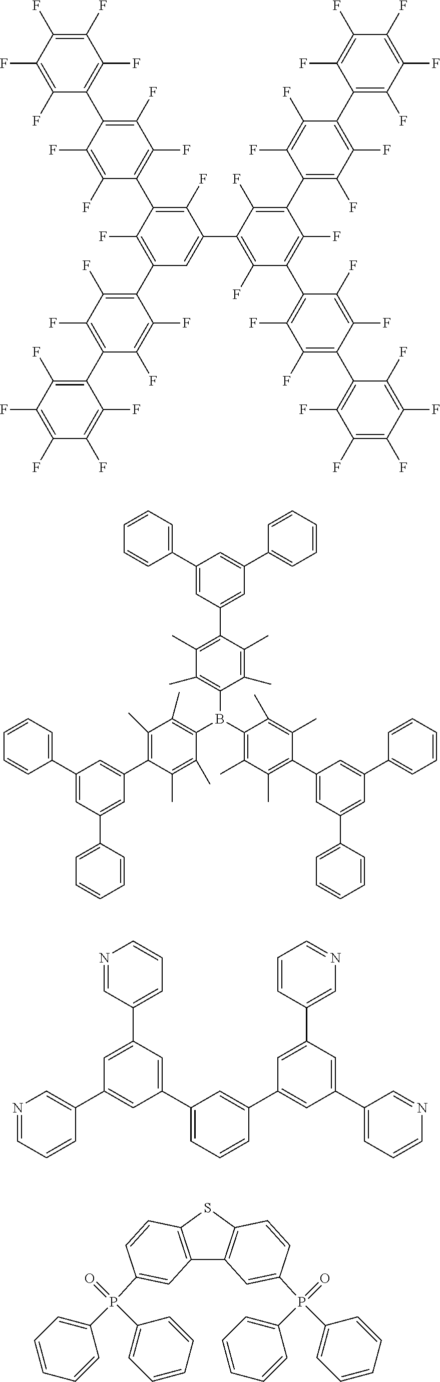

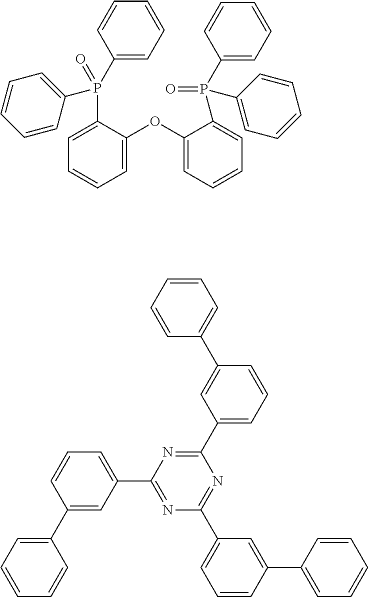 [SMILES: O=P(c1ccccc1)(c1ccccc1)c1ccccc1Oc1ccccc1P(=O)(c1ccccc1)c1ccccc1 ; c1ccc(-c2cccc(-c3nc(-c4cccc(-c5ccccc5)c4)nc(-c4cccc(-c5ccccc5)c4)n3)c2)cc1]

Next, preferred compounds for use as an electron transport material are mentioned below.

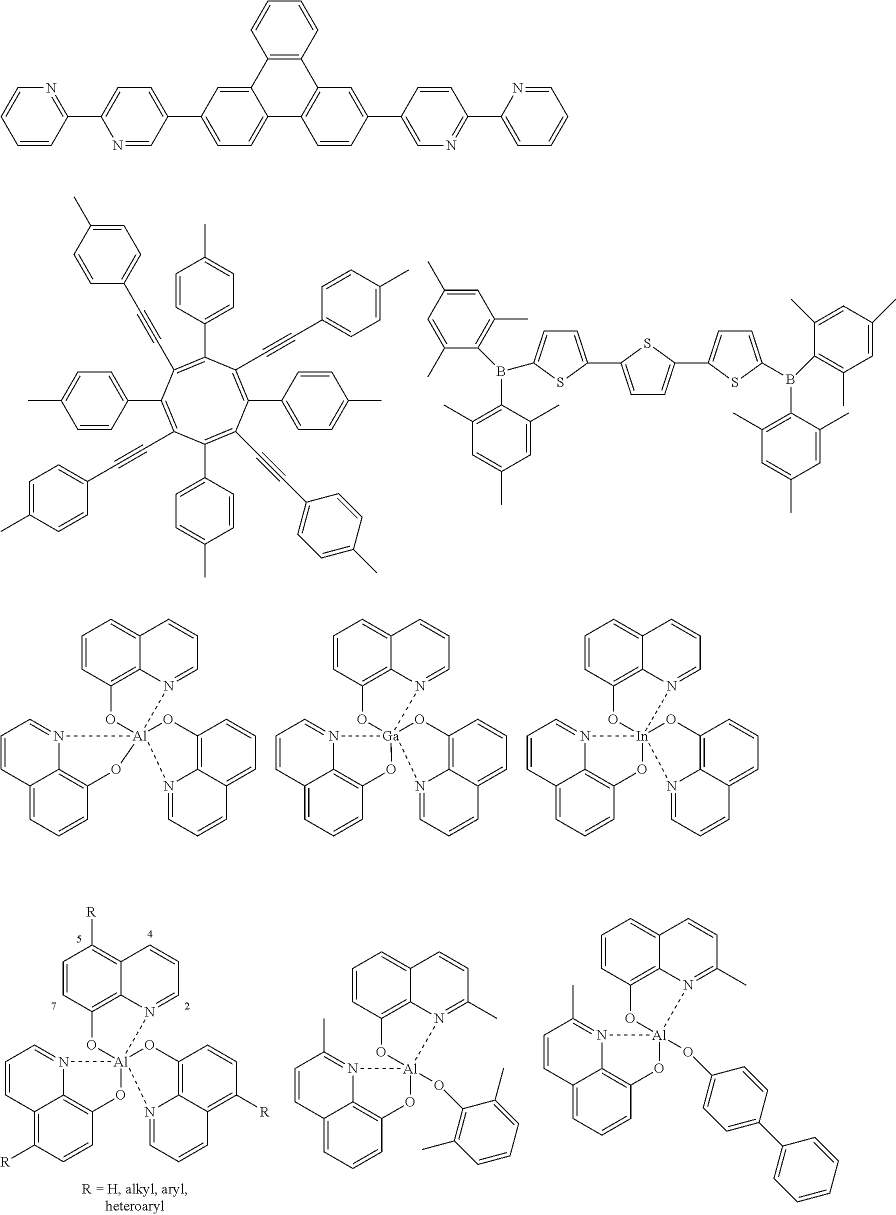

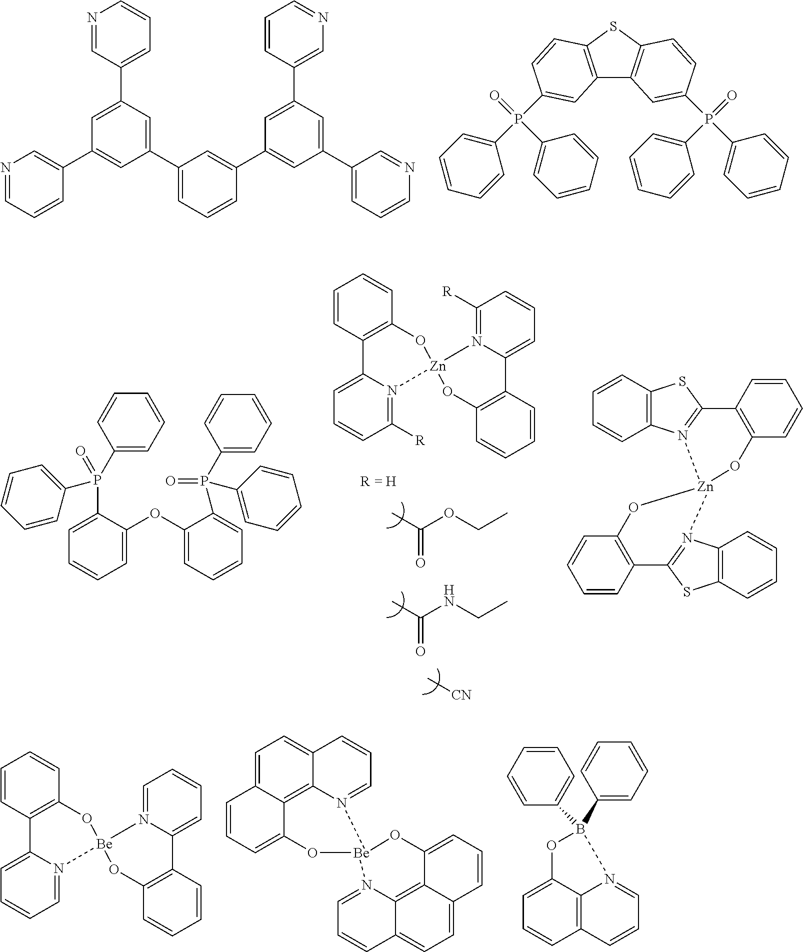

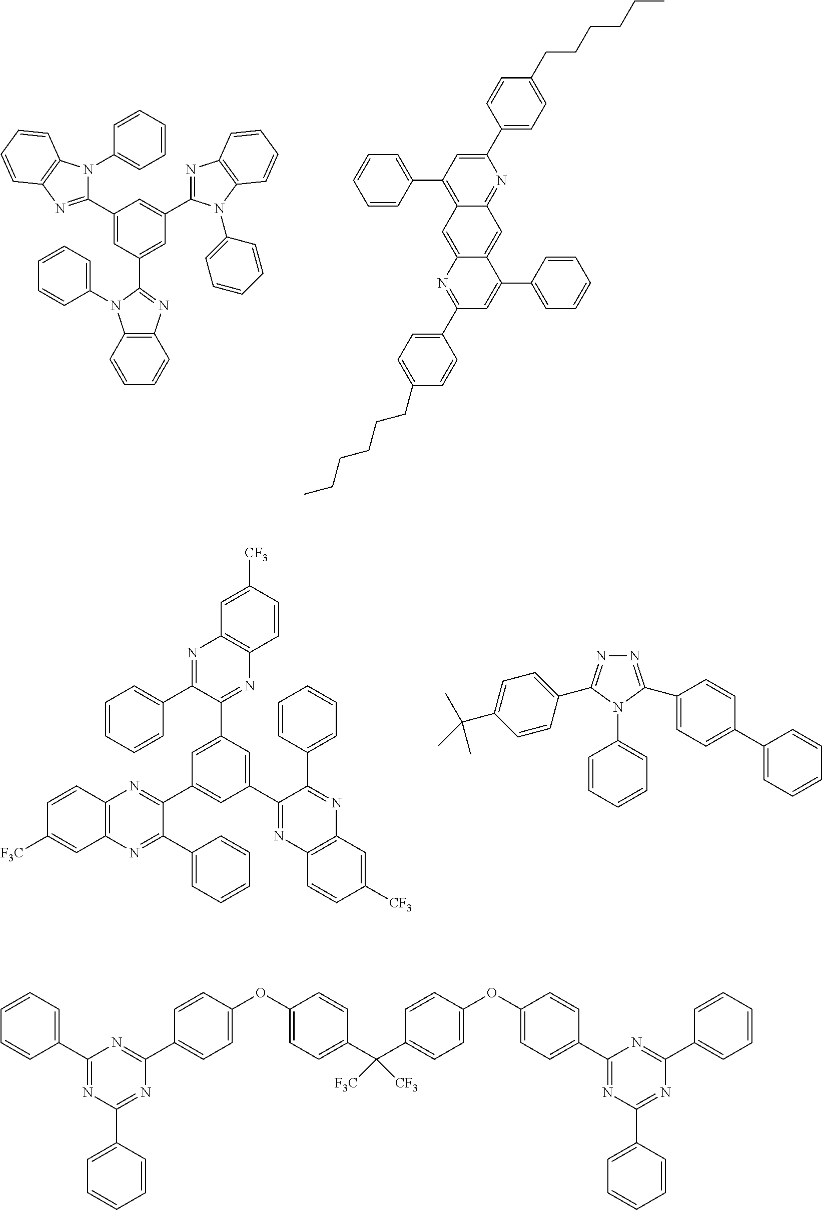 [SMILES: c1ccc(-n2c(-c3cc(-c4nc5ccccc5n4-c4ccccc4)cc(-c4nc5ccccc5n4-c4ccccc4)c3)nc3ccccc32)cc1 ; CCCCCCc1ccc(-c2cc(-c3ccccc3)c3cc4nc(-c5ccc(CCCCCC)cc5)cc(-c5ccccc5)c4cc3n2)cc1 ; FC(F)(F)c1ccc2nc(-c3cc(-c4nc5ccc(C(F)(F)F)cc5nc4-c4ccccc4)cc(-c4nc5ccc(C(F)(F)F)cc5nc4-c4ccccc4)c3)c(-c3ccccc3)nc2c1 ; CC(C)(C)c1ccc(-c2nnc(-c3ccc(-c4ccccc4)cc3)n2-c2ccccc2)cc1 ; FC(F)(F)C(c1ccc(Oc2ccc(-c3nc(-c4ccccc4)nc(-c4ccccc4)n3)cc2)cc1)(c1ccc(Oc2ccc(-c3nc(-c4ccccc4)nc(-c4ccccc4)n3)cc2)cc1)C(F)(F)F]

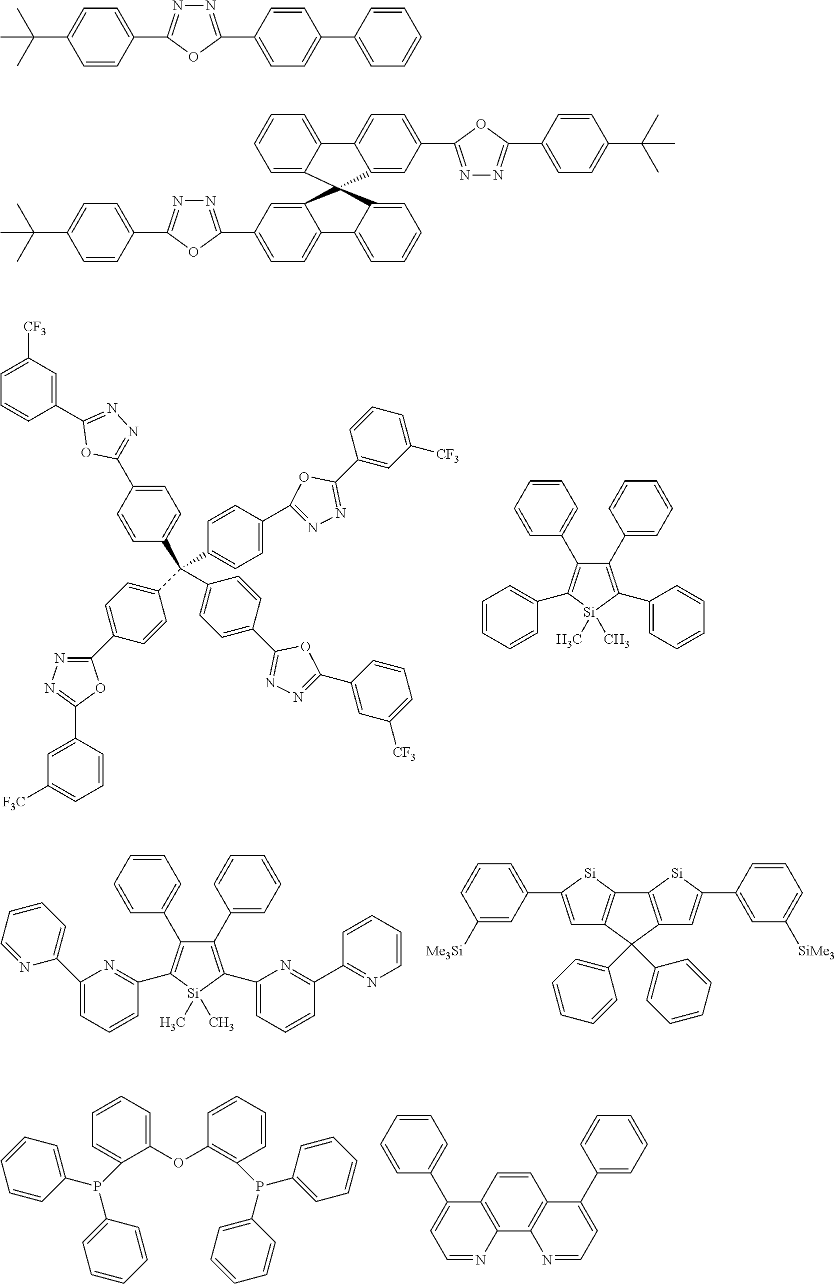

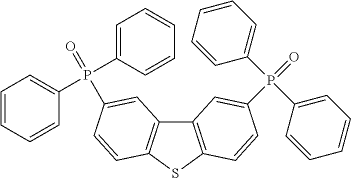 [SMILES: O=P(c1ccccc1)(c1ccccc1)c1ccc2sc3ccc(P(=O)(c4ccccc4)c4ccccc4)cc3c2c1]

Next, preferred compounds for use as an electron injection material are mentioned below.

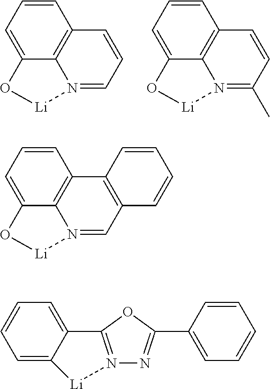

Further, preferred compounds for use as additional materials are mentioned below. For example, these are considered to be added as a stabilization material.

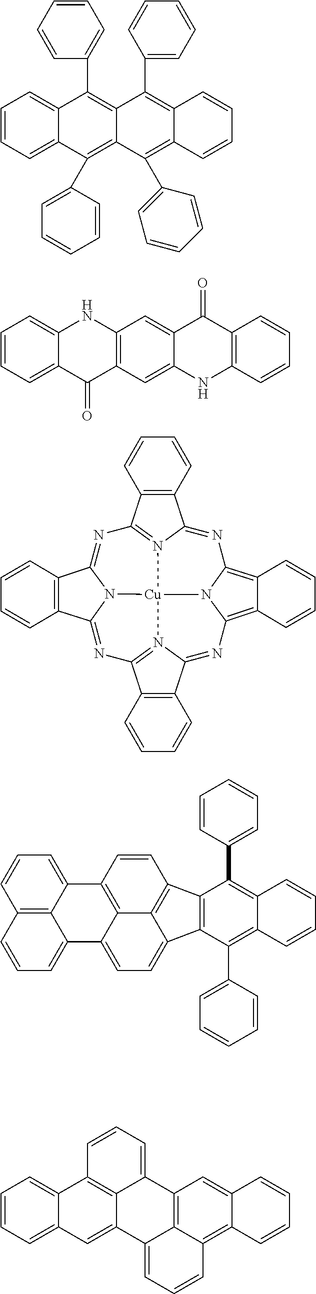

The organic electroluminescent device thus produced by the aforementioned method emits light on application of an electric field between the anode and the cathode of the device. In this case, when the light emission is caused by the excited singlet energy, light having a wavelength that corresponds to the energy level thereof may be confirmed as fluorescent light and delayed fluorescent light. When the light emission is caused by the excited triplet energy, light having a wavelength that corresponds to the energy level thereof may be confirmed as phosphorescent light. The normal fluorescent light has a shorter light emission lifetime than the delayed fluorescent light, and thus the light emission lifetime may be distinguished between the fluorescent light and the delayed fluorescent light.

On the other hand, the phosphorescent light may substantially not be observed with a normal organic compound such as the compound of the present invention at room temperature since the excited triplet energy is converted to heat or the like due to the instability thereof, and is immediately deactivated with a short lifetime. The excited triplet energy of the normal organic compound may be measured by observing light emission under an extremely low temperature condition.

The organic electroluminescent device of the invention may be applied to any of a single device, a device with plural structures disposed in an array, and a structure having anodes and cathodes disposed in an X-Y matrix. According to the present invention using the compound represented by the formula (1a) or the formula (1b) in a light emitting layer, an organic light emitting device having a markedly improved light emission efficiency can be obtained. The organic light emitting device such as the organic electroluminescent device of the present invention may be applied to a further wide range of purposes. For example, an organic electroluminescent display apparatus may be produced with the organic electroluminescent device of the invention, and for the details thereof, reference may be made to S. Tokito, C. Adachi and H. Murata, “Yuki EL Display” (Organic EL Display) (Ohmsha, Ltd.). In particular, the organic electroluminescent device of the invention may be applied to organic electroluminescent illumination and backlight which are highly demanded.

### EXAMPLES

The features of the present invention will be described more specifically with reference to Synthesis Examples and Examples given below. The materials, processes, procedures and the like shown below may be appropriately modified unless they deviate from the substance of the invention. Accordingly, the scope of the invention is not construed as being limited to the specific examples shown below. The light emission characteristics were evaluated using a fluorescence phosphorescence spectrophotometer (available from Horiba, Ltd., Fluoromax-4P), a small-size fluorescence lifetime measuring device (available from Hamamatsu Photonics K.K., Quantaurus-Tau C11367-01), a nitrogen cryostat (available from Oxford Instruments Inc., Optistat DN2), an absolute PL quantum yield measuring device (available from Hamamatsu Photonics K.K., C9920-02), an external quantum efficiency measuring device (available from Hamamatsu Photonics K.K., C9920-12), and a source meter (available from Keithley Instruments Inc., 2400 Series); the CIE chromaticity coordinate was measured using an absolute PL quantum yield measuring device (available from Hamamatsu Photonics K.K., C9920-02), an external quantum efficiency measuring device (available from Hamamatsu Photonics K.K., C9920-12); the thermal characteristics were evaluated using a differential thermal balance (available from NETZSCH Japan K.K., TG-DTA2000SEα) and a differential scanning calorimeter (available from Mettler Toledo Corp., DSC1). In the following, “instantaneous fluorescence” means a fluorescence having an emission lifetime of 50 ns or less in an emission transient decay curve, and “delayed fluorescence” means a fluorescence having an emission lifetime of 0.2 μs or more in an emission transient decay curve.

### Synthesis Examples

Compounds 1 to 3 were synthesized according to the following process.

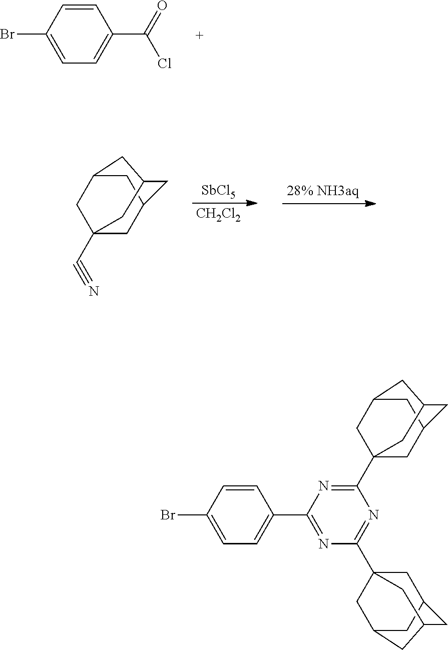

In an Ar atmosphere, 4-bromobenzoyl chloride (1.72 g, 7.84 mmol) and 1-adamantanecarbonitrile (2.50 g, 15.5 mmol) were dissolved in 10 ml of ultra-dehydrated methylene chloride and stirred in an ice bath at 0 to 5° C. for 30 minutes. A solution of antimony (V) chloride (1.0 M in methylene chloride) (8.5 mL, 8.5 mmol) was dropwisely added to the resultant solution, and gradually heated up to room temperature. The reaction mixture was kept stirred at room temperature for 1 hour, and refluxed at 45° C. for 38 hours. Subsequently, this was cooled down to room temperature and the greenish white solid was taken out through filtration and washed with methylene chloride. Next, this was gradually added to 55 mL of a 28% ammonia solution (0 to 5° C.), and stirred in an ice bath for 30 minutes. The mixture was warmed up to room temperature and stirred for 16.5 hours, and the resultant white solid was taken out through filtration and washed with distilled water. After dried, the solid was dissolved in toluene and filtered twice while hot. The filtrate was evaporated away, and the resultant residue was washed with a small amount of acetone to give a white solid of 2,4-di(adamantan-1-yl)-6-(4-bromophenyl)-1,3,5-triazine (0.10 g, 2.17 mmol, yield 28.1%).

¹H NMR (600 MHz, CDCl₃), δ(ppm): 8.47 (d, J=8.2 Hz, 2H), 7.62 (d, J=8.2 Hz, 2H), 2.08-2.13 (m, 18H), 1.79-1.84 (m, 12H).

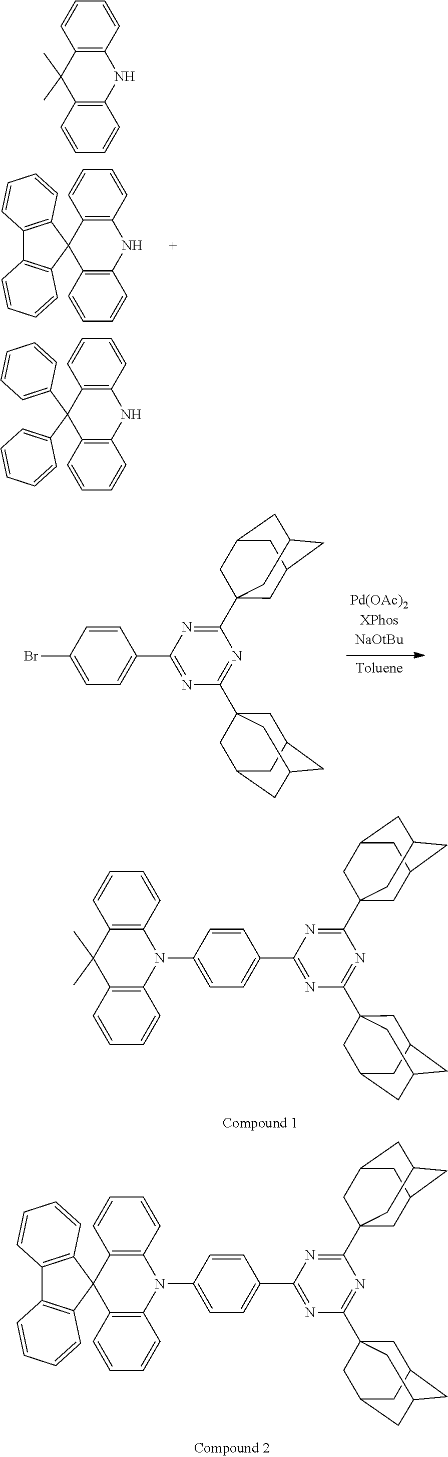

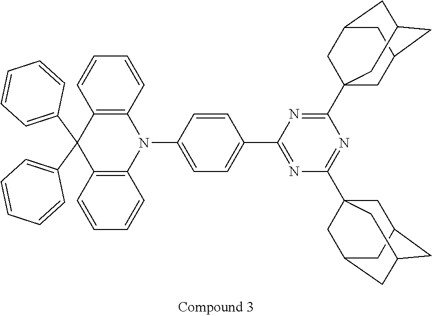 [SMILES: c1ccc(C2(c3ccccc3)c3ccccc3N(c3ccc(-c4nc(C56CC7CC(CC(C7)C5)C6)nc(C56CC7CC(CC(C7)C5)C6)n4)cc3)c3ccccc32)cc1]

9,9-Dimethyl-9,10-dihydroacridine (211 mg, 1.01 mmol), 2,4-di(adamantan-1-yl)-6-(4-bromophenyl)-1,3,5-triazine (456 mg, 0.30 mmol), sodium tert-butoxide (NaOtBu, 255 mg, 2.66 mmol), 2-dicyclohexylphosphino-2′,4′,6′-triisopropylbiphenyl (XPhos, 26.6 mg, 54.5 μmol) and palladium(II) acetate (Pd(OAc)₂, 8.4 mg, 37.4 μmol) wee dissolved in ultra-dehydrated toluene (10.0 mL), and stirred at 110° C. for 3.5 hours. After this was cooled to room temperature, the organic layer was extracted out with chloroform/salt water. Next, the organic layer was dried with sodium sulfate and filtered, and the filtrate was concentrated using a rotary evaporator. Next, using chloroform as an eluent, the crude product was filtered through silica gel to remove the residual catalyst. After the solvent was removed, the residue was dissolved in a small amount of chloroform, and a bad solvent acetone was added for precipitation and purification to give a white solid of a compound 1 as a moderate yield (451 mg, 79%).

Similarly, a compound 2 was obtained at a yield of 82.1%, and a compound 3 was at a yield of 82.0%.

### Compound 1:

¹H NMR (800 MHz, CDCl₃), δ(ppm): 8.84-8.86 (m, 2H), 7.46-748 (m, 4H), 6.93-6.97 (m, 4H), 6.33-6.34 (m, 2H) 2.16 (18H), 1.83 (12H) 1.72 (s, 6H).

¹³C-NMR (201 MHz, CDCl₃), δ (ppm): 184.35, 169.70, 144.67, 140.64, 136.86, 131.38, 131.33, 130.11, 126.37, 125.29, 120.71, 114.13, 41.29, 40.67, 36.82, 36.01, 31.30, 28.55.

APCI-MS (m/z): [M+H]⁺ calcd. for C₄₄H₄₉N₄, 633.3957; found, 633.3976.

Elemental analysis calcd. (%) for C₄₄H₄₈N₄: C, 83.50; H, 7.70; N, 8.89; found: C, 83.50; H, 7.64; N, 8.85.

### Compound 2:

¹H NMR (800 MHz, CD₂Cl₂), δ(ppm): 8.93-8.95 (m, 2H), 7.85 (d, J=7.6 Hz, 2H), 7.65-7.67 (m, 2H), 7.43 (d, J=7.5 Hz, 2H) 7.41 (td, J=7.4 Hz, 0.96 Hz, 2H), 7.30 (td, J=7.4 Hz, 0.96 Hz, 2H) 6.93 (ddd, J=8.5, 7.0, 1.5 Hz, 2H) 6.57-6.59 (m, 2H) 6.44 (dd, J=8.6 Hz, 0.88 Hz, 2H) 6.38 (dd, J=7.8 Hz, 0.80 Hz) 2.19 (d, J=2.9 Hz, 12H), 2.16 (m, 6H) 1.84-1.88 (m, 12H).

¹³C-NMR (201 MHz, CD₂Cl₂), δ(ppm) 184.14, 169.39, 156.25, 144.11, 140.80, 138.95, 136.95, 131.17, 131.04, 128.06, 127.41, 127.24, 126.99, 125.23, 124.51, 120.29, 119.74, 114.48, 56.48, 41.03, 40.36, 36.50, 28.43.

APCI-MS (m/z): [M+H]⁺ calcd. for C₅₄H₅₁N₄, 755.4114; found, 755.4103.

Elemental analysis calcd. (%) for C₅₄H₅₀N₄: C, 86.07; H, 6.74; N, 7.35; found: C, 85.90; H, 6.68; N, 7.41.

### Compound 3:

¹H NMR (800 MHz, CD₂Cl₂), δ (ppm): 8.74-8.76 (m, 2H), 7.25-7.30 (6H), 7.22-7.23 (m, 2H), 7.03-7.06 (m, 2H), 7.01-7.03 (m, 4H), 6.87-6.90 (m, 4H), 6.48 (m, 2H), 2.13-2.14 (18H), 1.81-1.85 (m, 12H).

¹³C-NMR (201 MHz, CD₂C₂), δ (ppm): 184.04, 169.35, 146.21, 143.92, 141.61, 136.63, 130.96, 130.59, 130.03, 129.73, 129.33, 127.31, 126.57, 126.01, 120.01, 113.84, 56.43, 40.97, 40.32, 36.48, 28.40.

APCI-MS (m/z): [M+H]⁺ calcd. for C₅₄H5₃N₄, 757.4270; found, 755.4280.

Elemental analysis calcd. (%) for C₅₄H₅₂N₄: C, 85.77; H, 6.97; N, 7.33; found: C, 85.68; H, 6.92; N, 7.40.

### (Evaluation 1) Preparation and Evaluation of Toluene Solutions of Compounds 1 to 3, and Production and Evaluation of Organic Photoluminescent Devices Using Compounds 1 to 3 and Compound H5 According to Coating Method

Measurement results of the glass transition temperature Ts and the thermal decomposition temperature T_{d} of the compounds 1 to 3 synthesized in Synthesis Examples are shown in Table 1. Here, the thermal decomposition temperature T_{d} is a temperature at which the weight loss of the compound is 5% on the TGA curve thereof.

A toluene solution of 10 mol/L of each of the compounds 1 to 3 was prepared.

Further, according to a spin coating method, a chloroform solution of 10 mg/mL of a mixture of any of the compounds 1 to 3 and a compound H5 shown below, as mixed in a weight ratio of 10 wt %, was drop-casted onto a quartz substrate and rotated at a rotating speed of 1000 rpm for 30 seconds to remove the solvent by drying, thereby forming a thin film to produce an organic photoluminescent device.

Each toluene solution of any of the prepared compounds 1 to 3, and each thin film of any of the compounds 1 to 3 and the compound 115 were individually irradiated with a 330 nm excitation light to measure the emission maximum wavelength. Before and after Ar bubbling, the photoluminescent quantum yield (PL quantum yield) of each toluene solution with a 369 nm, 379 nm or 350 nm excitation light was measured; and in a nitrogen atmosphere, the photoluminescent quantum yield (PL quantum yield) of each thin film with a 302 nm, 303 nm or 280 nm excitation light was measured. The measurement results are shown in Table 1.

For comparison, the glass transition temperature T_{g} and the thermal decomposition temperature T_{d} of the following comparative compound A were measured under the same condition. An emission maximum wavelength of a toluene solution of the comparative compound A (concentration 10⁻⁵ mol/L) was also measured. The result is also shown in Table 1.

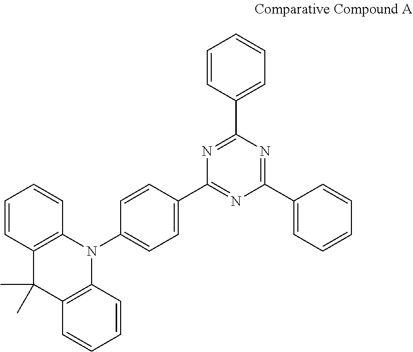 [SMILES: CC1(C)c2ccccc2N(c2ccc(-c3nc(-c4ccccc4)nc(-c4ccccc4)n3)cc2)c2ccccc21]

TABLE 1

| | Toluene Solution | | | Thin Film | | | Thermal Characteristics | |
|---|---|---|---|---|---|---|---|---|
| | Maximum | | | | Maximum | PL | Glass | Thermal |
| | Emission | PL Quantum | | | Emission | Quantum | Transition | Decom- |
| | Wave- | Yield ϕ_{PL} (%) | | Host | Wave- | Yield ϕ_{PL} | Temper- | position |
| | length | before Ar | after Ar | Com- | length | (%) in | ature | Temperature |
| Compound | λ_{MAX} | bubbling | bubbling | pound | λ_{MAX} | nitrogen | T_{g} | T_{d} |
| No. | (nm) | (%) | (%) | No. | (nm) | (%) | (° C.) | (° C.) |
| Compound | 469 | 18 | 45 | Com- | 480 | 88 | 147 | 387 |
| 1 | | | | pound | | | | |
| Compound | 454 | 20 | 40 | H5 | 469 | 86 | 207 | 421 |
| 2 | | | | | | | | |
| Compound | 451 | 15 | 34 | | 460 | 70 | 186 | — |
| 3 | | | | | | | | |
| Comparative | 494 | — | — | — | — | — | 91 | 305 |
| Compound | | | | | | | | |
| A | | | | | | | | |

As shown in Table 1, each toluene solution of any of the compounds 1 to 3, and each thin film of any of the compounds 1 to 3 and the compound H5 all had a high PL quantum yield. In addition, the compounds 1 to 3 had a higher glass transition temperature T_{g} and a higher thermal decomposition temperature T_{d} than the comparative compound A, and are therefore known to be excellent in thermal stability.

### (Evaluation 2) Production and Evaluation of Organic Photoluminescent Devices Using Compound 2 and Various Host Compounds According to Coating Method

According to a spin coating method, a chloroform solution of 10 mg/mL of a mixture of the compound 2 and a host compound shown in Table 2, as prepared so that the concentration of the compound 2 therein could be 10 to 100 wt %, was drop-casted onto a quartz substrate and rotated at a rotating speed of 1000 rpm for 30 seconds to remove the solvent by drying, thereby forming a thin film to produce an organic photoluminescent device.

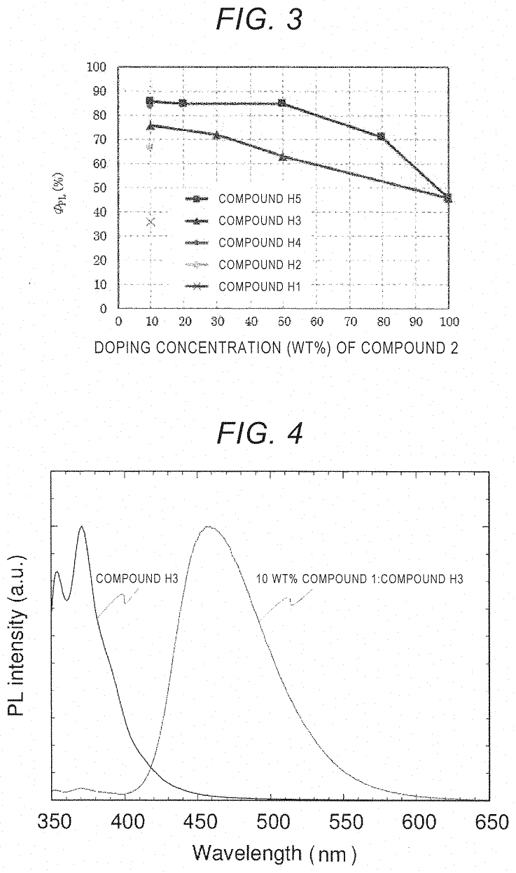

Emission spectra of thin films in which the concentration of the compound 2 was 10 wt % are shown in FIG. 2, and the photoluminescent quantum yield (PL quantum yield) and the CIE chromaticity coordinate (x, y) thereof are shown in Table 2. A relationship between the doping concentration of the compound 2 in each thin film and the PL quantum yield is shown in FIG. 3. Here, the wavelength of the excitation light used in measurement of the emission spectrum, the PL quantum yield and the CIE chromaticity coordinate was 297 nm for the thin film in which the compound H1 was used as the host compound, 287 nm for the thin film in which the compound H2 was used as the host compound, 280 nm for the thin film in which the compound H3 was used as the host compound, 273 nm for the thin film in which the compound H4 was used as the host compound, and 280 nm for the thin film in which the compound H5 was used as the host compound. In FIG. 2, “Host Compound H1”, “Host Compound H3” to “Host Compound H5” mean that the indicated compound was used as the host compound in the thin film and the thin film contained 10 wt % of the compound 2. In FIG. 3, “Host Compound H1” to “Host Compound H5” mean that the indicated compound was used as the host compound in the thin film and concentration of the compound 2 therein was varied.

TABLE 2

| | Host | PL Quantum Yield (%) | | CIE |
|---|---|---|---|---|
| Compound | Compound | Under nitrogen | Under air | Chromaticity |
| No. | No. | atmosphere | atmosphere | Coordinate |
| Compound | Compound H1 | 36 | 32 | (0.16, 0.13) |
| 2 | Compound H2 | 67 | 38 | (0.15, 0.13) |
| | Compound H3 | 76 | 36 | (0.15, 0.13) |
| | Compound H4 | 84 | 57 | (0.16, 0.18) |
| | Compound H5 | 86 | 67 | (0.16, 0.22) |

As shown in Table 2, all the thin films had high PL quantum yields, and especially the thin film using any of the compounds H3 to H5 as the host compound had a high PL quantum yield. From each thin film, a good blue light having a CIE chromaticity coordinate of x≤0.16 and y≤0.22 was observed, and in particular, the thin films using any of the compounds H1 to H3 as the host compound gave an extremely good blue light having a CIE chromaticity coordinate of x≤0.16 and y≤0.13.

### (Evaluation 3) Production and Evaluation of Organic Photoluminescent Device Using Compound 1 and Compound H3 According to Coating Method

According to a spin coating method, a chloroform solution of 10 mg/mL of a mixture of the compound 1 and a compound H3 shown below, as mixed in a weight ratio of 10 wt %, was drop-casted onto a quartz substrate and rotated at a rotating speed of 1000 rpm for 30 seconds to remove the solvent by drying, thereby a thin film was obtained and used as an organic photoluminescent device.

Apart from this, the above-mentioned solution was drop-casted onto a quartz substrate and dried in vacuum to form a cast film in which the concentration of the compound 1 was 10 wt %, which was used as an organic photoluminescent device.

Emission characteristics with a 300 nm excitation light of the thin film (cast film) of the compound 1 and the compound H3 were evaluated. FIG. 4 shows an emission spectrum of the cast film and, for comparison, an emission spectrum of a thin film formed by vapor deposition of the compound H3 alone. In FIG. 4, “10 wt % compound 1: compound H3” means a cast film of the compound 1 and the compound H3; and “compound H3” means a thin film of the compound H3 alone formed by vapor deposition.

The cast film of the compound 1 and the compound H3 had a PL quantum yield of 93% and a CIE chromaticity coordinate (x, y) of (0.15, 0.18) and therefore had good emission characteristics. From this, it is known that the compound 1 can be formed into a film excellent as a light emitting layer according to a coating method.

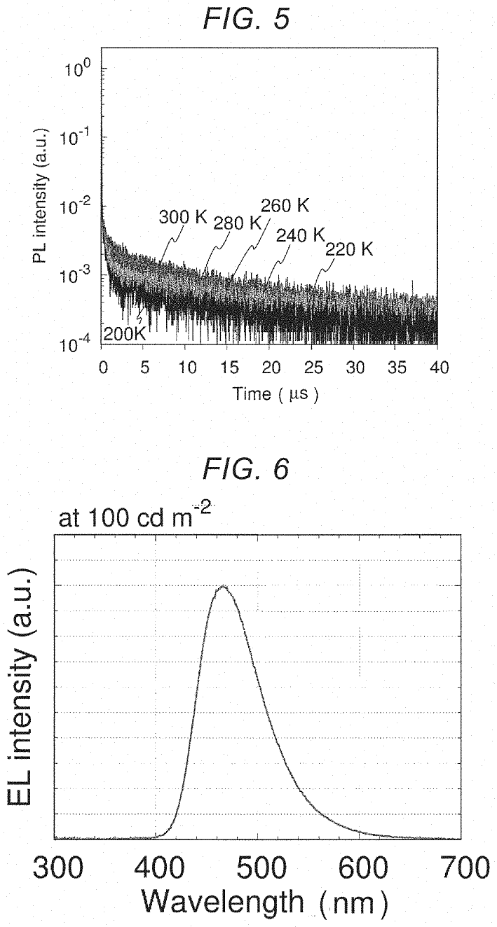

In addition, the transient decay curve of light emission, as measured at 200 K, 220 K, 240 K, 260 K, 280 K or 300 K, of the spin-coating film of the compound 1 and the compound H3 is shown in FIG. 5. The emission detection wavelength is 457 nm. The emission lifetime measured at 300 K was 14.5 ns in instantaneous fluorescence and 9.1 μs in delayed fluorescence; and the PL quantum yield was 62.2% in instantaneous fluorescence and 30.8% in delayed fluorescence, and the total thereof was 93%. The measurement results confirm that the compound 1 is a thermal activation type delayed fluorescent material.

### (Evaluation 4) Evaluation of Organic Electroluminescent Device Having a Light Emitting Layer Formed According to Coating Method

On a glass substrate having, as formed thereon, an anode of indium tin oxide (ITO) having a thickness of 50 nm, each thin film was layered according to a spin coating method. First, on ITO, an aqueous solution of 50 mass % PEDOT:PSS (available from Heraeus Corp., CH8000) was drop-casted and spin-coated thereon at 500 rpm for 1 second, at 4000 rpm for 10 seconds and at 500 rpm for 1 second in that order, and then heat-treated at 150° C. for 10 minutes to thereby form a PEDOT:PSS film having a thickness of 45 nm. Next, on the PEDOT:PSS film, a 1,2-dichlorobenzene solution of 10 mass % PVK (poly(9-vinylcarbazole), available from Sigma Aldrich Corp., weight-average molecular weight, about 1,100,000) was drop-casted and spin-coated thereon at 2000 rpm for 30 seconds, and thereafter heat-treated at 120° C. for 10 minutes to form a PVK film having a thickness of 15 nm. Next, on the PVK film, a toluene solution of a mixture of the compound 1 and the compound H3, as mixed in a ratio by mass of 10/90 and dissolved to have a total concentration of 10 mg/mL, was drop-casted and spin-coated thereon at 2200 rpm for 30 seconds, and then heat-treated at 100° C. for 10 minutes to thereby form a film of a light emitting layer having a concentration of the compound 1 of 10 mass % and having a thickness of 35 nm.

Next, on the light emitting layer, a film of a compound H4 was formed to have a thickness of 5 nm according a vacuum evaporation method at a vacuum degree of less than 10⁻⁴ Pa, then a film of TPBi was formed to have a thickness of 50 nm, and on this, Liq was formed to have a thickness of 1 nm. Subsequently, according to a vacuum evaporation method at a vacuum degree of less than 10⁻⁴ Pa, Al having a thickness of 80 nm was formed to be a cathode.

According to the above-mentioned process, an organic electroluminescent device having a layer configuration of ITO (50 nm)/PEDOT:PSS (45 nm)/PVK (15 nm)/10 mass % compound 1, compound H3 (35 nm)/compound H4 (5 nm)/TPBi (50 nm)/Liq (1 nm)/Al (80 nm) was produced. In the layer configuration, “I” indicates a layer boundary, and the numerical value in each parenthesis indicates a layer thickness.

An emission spectrum of the thus-produced organic electroluminescent device is shown in FIG. 6. The organic electroluminescent device had a maximum external quantum efficiency EQE_{MAX} of 22.1%, an external quantum efficiency at 100 cd/m² of 13.9%, and a CIE chromaticity coordinate (x, y) of (0.15, 0.19), and realized a high emission efficiency and an extremely good blue light emission.

### (Evaluation 5) Evaluation of Organic Electroluminescent Device Having a Light Emitting Layer Formed According to Vacuum Evaporation Method

On a glass substrate having, as formed thereon, an anode of indium tin oxide (ITO) having a thickness of 50 nm, each thin film was layered at a vacuum degree of less than 10⁻⁴ Pa according to a vapor evaporation method at a vacuum degree of less than 10⁻⁴ Pa. First, on ITO, TAPC having a thickness of 60 nm was formed, and on it, a layer of the compound H2 having a thickness of 10 nm was formed. Subsequently, the compound 1 and the compound H3 were co-evaporated from different evaporation sources to form a layer having a thickness of 20 nm as a light emitting layer. At this time, the concentration of the compound 1 was 10 mass %. Next, on the light emitting layer, the compound H5 was layered in a thickness of 10 nm, and further on it, BmPyPhB was formed in a thickness of 30 nm. Subsequently, Liq was formed in a thickness of 1 nm, and on it, Al was vapor-deposited in a thickness of 80 nm to be a cathode.

According to the above-mentioned process, an organic electroluminescent device (device 1) having a layer configuration of ITO (50 nm)/TAPC (60 nm)/compound H2 (10 nm)/10 mass % compound 1, compound H3 (20 nm)/compound H5 (10 nm)/BmPyPhB (30 nm)/Liq (1 nm)/Al (80 nm) was produced. In the layer configuration, “/” indicates a layer boundary, and the numerical value in each parenthesis indicates a layer thickness.

Apart from this, an organic electroluminescent device (device 2) was produced in the same manner as that for the device 1 except that the concentration of the compound 1 in the light emitting layer was 20 mass %; and further an organic electroluminescent device (device 3) was produced in the same manner as that for the device 1 except that the light emitting layer was formed using mCBP in place of the compound H3. The layer configuration of each device is shown below.

Device 2: ITO (50 nm)/TAPC (60 nm)/compound H2 (10 nm)/20 mass % compound 1, compound H3 (20 nm)/compound H5 (10 nm)/BmPyPhB (30 nm)/Liq (1 nm)/Al (80 nm)
Device 3: ITO (50 nm)/TAPC (60 nm)/compound H2 (10 nm)/10 mass % compound 1, mCBP (20 nm)/compound H5 (10 nm)/BmPyPhB (30 nm)/Liq (1 nm)/Al (80 nm)

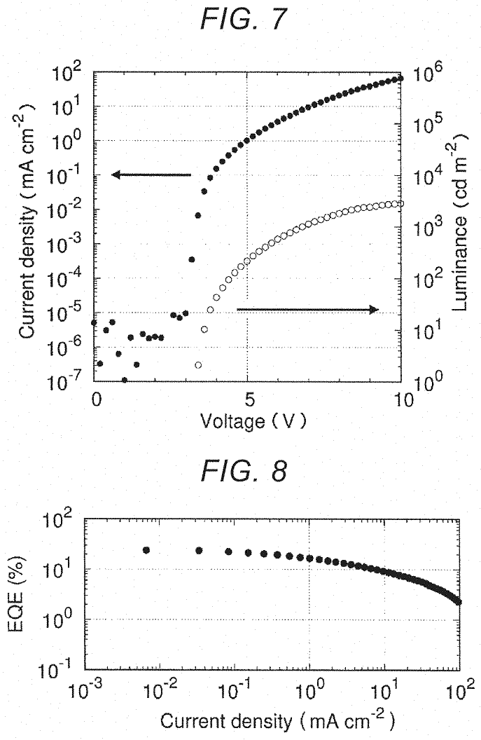

FIG. 7 shows a graph of current density-voltage-luminance characteristic of the device 1 produced herein, and FIG. 8 shows a graph of external quantum efficiency EQE-current density characteristic thereof. The device 1 had an emission maximum wavelength λ_{MAX} of 459 nm, a CIE chromaticity coordinate (x, y) of (0.15, 0.16), a maximum luminance L_{MAX} of 2882 cdm⁻², and a maximum external quantum efficiency EQE_{MAX} of 23.8%. The device 2 had an emission maximum wavelength λ_{MAX} of 465 nm, a CIE chromaticity coordinate (x, y) of (0.15, 0.19), a maximum luminance L_{MAX} of 5281 cdm⁻², and a maximum external quantum efficiency EQE_{MAX} of 22.3%. The device 3 had an emission maximum wavelength λ_{MAX} of 472 nm, a CIE chromaticity coordinate (x, y) of (0.16, 0.24), a maximum luminance L_{MAX} of 6975 cdm⁻², and a maximum external quantum efficiency EQE_{MAX} of 19.9%.

As described above, the devices 1 to 3 all had a high external quantum efficiency. In particular, the maximum external quantum efficiency EQE_{MAX} of the device 1 was high, and the CIE chromaticity coordinate y thereof was 0.16 or less, and the device 1 realized a deep blue emission. The device 2 where the concentration of the compound 1 increased and the device 3 where the host material was changed to a popular substance, mCBP attained a high luminance (5281 cdm⁻² of device 2 and 6975 cdm⁻² of device 3) while maintaining a high external quantum efficiency.

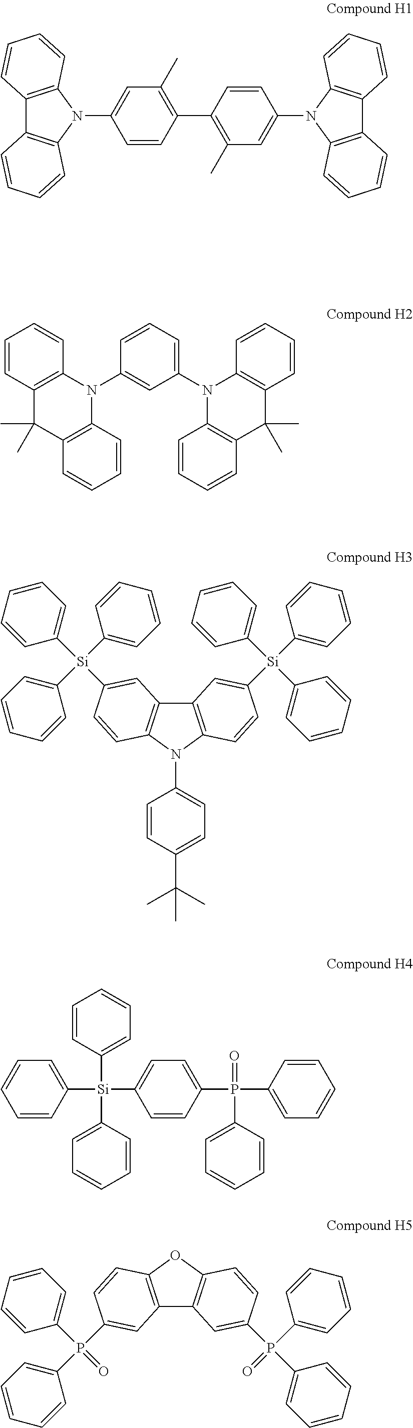

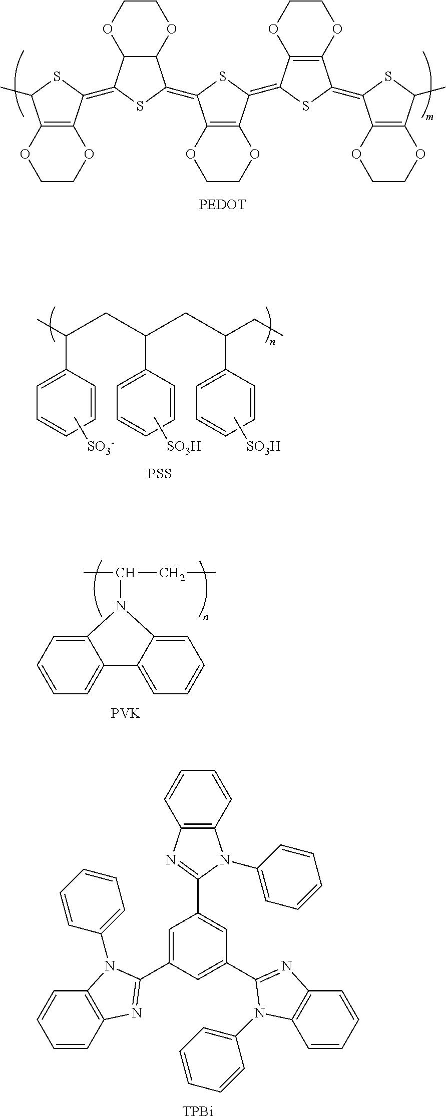

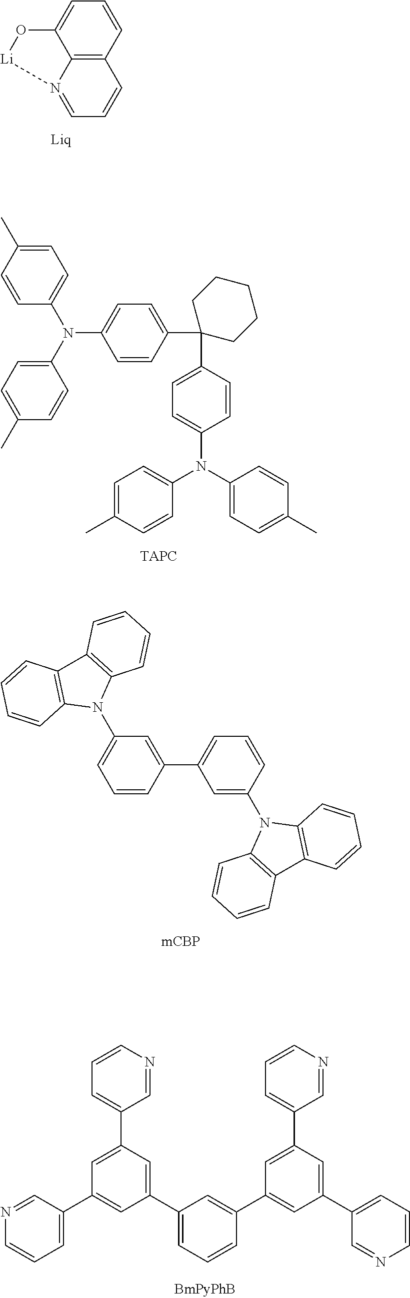

### INDUSTRIAL APPLICABILITY

The compound of the present invention can be readily formed into a film according to a coating method, and is useful as a light emitting material as having high thermal stability. Consequently, the compound of the present invention can be practically used as a light emitting material for organic light emitting devices such as organic electroluminescent devices. Accordingly, the industrial applicability of the present invention is great.

### REFERENCE SIGNS LIST

1 Substrate
2 Anode
3 Hole Injection Layer
4 Hole Transport Layer
5 Light emitting Layer
6 Electron Transport Layer
7 Cathode

1. A compound represented by the following formula (1a) or formula (1b): 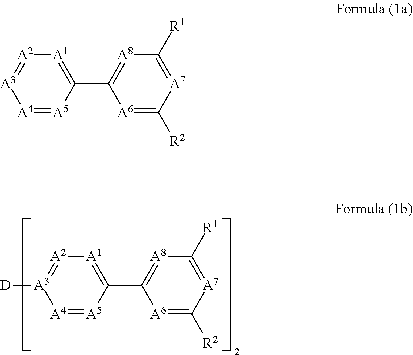
wherein R¹ and R² each independently represent an aliphatic group, and at least one represents a polycyclic aliphatic group; A¹ to A⁵ each independently represent N or C—R³, and R³ represents a hydrogen atom or a substituent, provided that at least one of A¹ to A⁵ is C—R³ and R³ is a donor group; A⁶ to A each independently represent N or C—R⁴, and R⁴ represents a hydrogen atom or an alkyl group, provided that at least one of A⁶ to A⁸ is N; and wherein D represents a donor-like linking group, and two R¹'s, two R²'s, and two A¹'s to two A⁸'s existing in the formula each may be the same as or different from each other.

2. The compound according to claim 1, wherein the polycyclic aliphatic group is a cage-structure aliphatic group.

3. The compound according to claim 1, wherein R¹ and R² are both a polycyclic aliphatic group.

4. The compound according claim 1, wherein at least one R³ has a structure represented by the following formula (2): 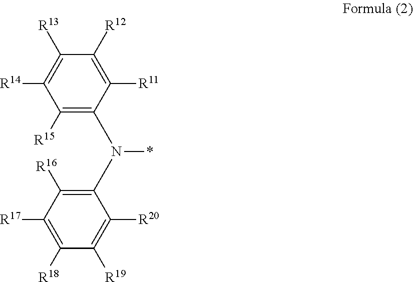
wherein R¹¹ to R²⁰ each independently represent a hydrogen atom or a substituent; R¹¹ and R¹², R¹² and R¹³, R¹³ and R¹⁴, R¹⁴ and R¹⁵, R¹⁵ and R¹⁶, R¹⁶ and R¹⁷, R¹⁷ and R¹⁸, R¹⁸ and R¹⁹, and R¹⁹ and R²⁰ each may bond to each other to form a cyclic structure; and * represents a bonding position.

5. The compound according to claim 4, wherein the structure represented by the formula (2) has a structure represented by any of the following formulae (3) to (7): 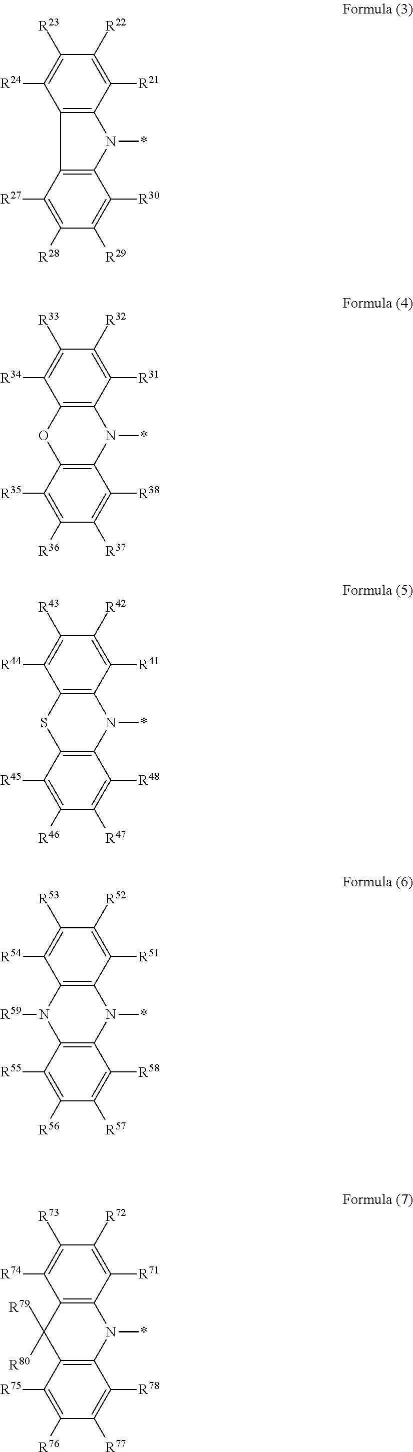
wherein R²¹ to R²⁴, R²⁷ to R³⁸, R⁴¹ to R⁴⁸, R⁵¹ to R⁵⁹, and R⁷¹ to R⁸⁰ each independently represent a hydrogen atom or a substituent; R²¹ and R²², R²² and R²³, R²³ and R²⁴, R²⁷ and R²⁸, R²⁸ and R²⁹, R²⁹ and R³⁰, R³¹ and R³², R³² and R³³, R³³ and R³⁴, R³⁵ and R³⁶, R³⁶ and R³⁷, R³⁷ and R³⁸, R⁴¹ and R⁴², R⁴² and R⁴³, R⁴³ and R⁴⁴, R⁴⁵ and R⁴⁶, R⁴⁶ and R⁴⁷, R⁴⁷ and R⁴⁸, R⁵¹ and R⁵², R⁵² and R⁵³, R⁵³ and R⁵⁴, R⁵⁵ and R⁵⁶, R⁵⁶ and R⁵⁷, R⁵⁷ and R⁵⁸, R⁵⁴ and R⁵⁹, R⁵⁵ and R⁵⁹, R⁷¹ and R⁷², R⁷² and R⁷³, R⁷³ and R⁷⁴, R⁷⁵ and R⁷⁶, R⁷⁶ and R⁷⁷, R⁷⁷ and R⁷⁸, and R⁷⁹ and R⁸⁰ each may bond to each other to form a cyclic structure; and * represents a bonding position.

6. The compound according to claim 1, wherein, when A² is C—R³ (where R³ is a donor group), both A¹ and A³ are not N, when A³ is C—R³ (where R³ is a donor group), both A² and A⁴ are not N, and when A⁴ is C—R³ (where R³ is a donor group), both A³ and A⁵ are not N.

7. The compound according to claim 1, wherein A¹ to A⁵ are all C—R³.

8. The compound according to claim 1, wherein A⁶ to A⁸ are all N.

9. The compound according to claim 1, which is composed of only a carbon atom, a nitrogen atom and a hydrogen atom.

10. The compound according to claim 1, of which the difference ΔE_{ST} between the lowest excited singlet energy level and the lowest excited triplet energy level is 0.3 eV or less.

11. A light emitting material comprising a compound of claim 1.

12. A delayed fluorescent material comprising a compound of claim 1.

13. An organic light emitting device comprising a compound of claim 1.

14. The organic light emitting device according to claim 13, which is an organic electroluminescent device.

15. The organic light emitting device according to claim 13, which contains the compound in the light emitting layer therein.

16. The organic light emitting device according to claim 15, wherein the light emitting layer contains a host material.

17. The organic light emitting device according to claim 13, which emits delayed fluorescence.

18. The organic light emitting device according to claim 13, which emits light such that the chromaticity coordinate x in the CIE-XYZ color coordinate system is 0.16 or less and y is 0.22 or less.
